(19) United States
(12) Patent Application Publication
(10) Pub. No.: US 2026/0351414 A1
(43) Pub. Date: Nov. 5, 2026

(54) SYSTEMS AND METHODS FOR MEASURING AGING AND PREDICTING AND SLOWING CHRONIC DISEASE

(71) Applicants: Duke University, Durham, NC (US); President and Fellows of Harvard College, Cambridge, MA (US)

(72) Inventors: Ahmad HARIRI, Durham, NC (US); Terrie MOFFITT, Durham, NC (US); Avshalom CASPI, Durham, NC (US); Annchen KNODT, Durham, NC (US); Ethan WHITMAN, Durham, NC (US); Maxwell Lorenz ELLIOTT, Cambridge, MA (US)

(21) Appl. No.: 19/667,490

(22) Filed: May 4, 2026

Related U.S. Application Data
(60) Provisional application No. 63/798,914, filed on May 2, 2025.

(51) Int. Cl. G16H 50/20; G06T 7/00; G06T 7/62; G16H 20/10; G16H 20/30; G16H 20/60; G16H 20/70; G16H 50/30; G16H 50/70

(52) U.S. Cl. CPC G16H 50/20; G06T 7/0012; G06T 7/62; G16H 20/10; G16H 20/30; G16H 20/60; G16H 20/70; G16H 50/30; G16H 50/70; G06T 2207/10088; G06T 2207/20081; G06T 2207/30016

(57) ABSTRACT

Disclosed herein are systems and methods for providing a metric of aging of a subject, including: acquiring brain imaging of the subject; extracting a plurality of structural measures from the brain imaging data; applying a trained model to the plurality of structural measures to determine a pace of aging score; comparing the pace of aging score of the subject to a normative reference distribution to determine a centile score, wherein the normative reference comprises a plurality of pace of aging scores corresponding to a plurality of control subjects; and outputting the metric of health, in which the metric of health comprises the pace of aging score and the centile score of the subject.

### CROSS REFERENCE TO RELATED APPLICATIONS

This application claims priority benefit from U.S. Application Ser. No. 63/798,914, filed May 2, 2025. The entirety of which is incorporated herein by reference.

### STATEMENT REGARDING FEDERALLY SPONSORED RESEARCH

This invention was made with government support under AG032282, AG049789, AG068432, and AG073207 awarded by the National Institutes of Health. The government has certain rights in the invention.

### BACKGROUND

Aging is the gradual, progressive and correlated decline of multiple organ systems over decades. Longitudinal studies provide evidence for substantial individual variation in the rate of aging (e.g., people born in the same year can age slower or faster than their peers). Aging clocks, such as epigenetic clocks, predict a subject's relative age, but are limited by the data on which they are trained to make the predictions. Emerging evidence suggests that aging clocks trained on longitudinal data may provide greater insights to a subject's relative age and potential diseases.

### SUMMARY

The present disclosure provides systems and methods for measuring the rate of longitudinal aging derived from cross-sectional brain MRI, termed Dunedin Pace of Aging Calculated from Neuroimaging or DunedinPACNI. Faster DunedinPACNI predicted participants' cognitive impairment, accelerated brain atrophy, and conversion to diagnosed dementia. Underscoring close links between longitudinal aging of the body and brain, faster DunedinPACNI also predicted physical frailty, poor health, future chronic diseases, and mortality in older adults.

Provided here are methods, systems, and computer readable media for estimating a metric of health of a subject. The method may include: acquiring brain imaging of the subject; extracting a plurality of structural measures from the brain imaging data; applying a trained model to the plurality of structural measures to determine a pace of aging score (e.g., a DunedinPACNI score); comparing the pace of aging score of the subject to a normative reference distribution to determine a centile score, in which the normative reference comprises a plurality of pace of aging scores corresponding to a plurality of control subjects; and outputting the metric of health, in which the metric of health comprises the pace of aging score and the centile score of the subject.

Also provided are methods of predicting a subject's likelihood of developing a disorder based on the subject's centile score and pace of aging including: acquiring brain imaging of the subject; extracting a plurality of structural measures from the brain imaging data; applying a trained model to the plurality of structural measures to determine the pace of aging score; comparing the pace of aging score of the subject to a normative reference distribution to determine a centile score, in which the normative reference distribution comprises a plurality of pace of aging scores corresponding to a plurality of controlled subjects; and outputting a prediction of the subject's likelihood of developing the disorder, in which if the centile score is over 50 or the pace of aging is ahead of age and sex matched controls the subject is at increased likelihood of developing the disorder. The methods may further include administering a recommended intervention to mitigate risk of developing the disorder or delay onset or progression of a chronic disorder.

### BRIEF DESCRIPTION OF THE DRAWINGS

The patent or patent application file contains at least one drawing in color. Copies of this patent or patent application publication with color drawings will be provided by the Office upon request and payment of the necessary fee.

FIGS. 1A-1D show a schematic overview of the study methods. FIG. 1A. Plot of mean scores for all 19 biomarkers comprising the Pace of Aging across four waves of observation at ages 26, 32, 38 and 45 years in the Dunedin Study (housed at the University of Otago). Hypothetical individual trajectories are shown for people with relatively slow, average and fast Pace of Aging from ages 26 to 45 years. FIG. 1B, Distribution of Pace of Aging scores in Dunedin Study members at age 45. Warmer colors represent a faster Pace of Aging; cooler colors represent a slower Pace of Aging. FIG. 1C, A single T1-weighted MRI scan collected from 860 Dunedin Study members at age 45 years was used to train an elastic net regression model to predict the Pace of Aging. We call the resulting measure DunedinPACNI. FIG. 1D, Regression weights from the DunedinPACNI model developed in the Dunedin Study were applied to T1-weighted MRI scans collected in the ADNI and UKB datasets to derive DunedinPACNI scores. Those scores were then related to aging-related phenotypes. AL, attachment loss; Apo, apolipoprotein; BMI, body mass index; FEV1, forced expiratory volume in 1 s; eGFR, estimated glomerular filtration rate; HbAlc, glycated hemoglobin; HDL, high-density lipoprotein; hsCRP, high sensitivity C-reactive protein; VO₂max, maximal oxygen uptake.

FIGS. 2A-2B show DunedinPACNI model validation. FIG. 2A, In-sample correlation between Pace of Aging and DunedinPACNI. FIG. 2B, Comparison of absolute effect sizes for associations between DunedinPACNI and Pace of Aging with physical functioning, cognition and subjective aging measures in 860 members of the Dunedin Study. Effects are presented as standardized β coefficients with the error bars as the 95% CIs.

FIGS. 3A-3D show DunedinPACNI predicts cognition, cognitive impairment and conversion to dementia. FIG. 3A, FIG. 3B, Cross-sectional associations between DunedinPACNI and cognitive test scores in ADNI (FIG. 3A) and UKB (FIG. 3B). FIG. 3A, 3B, Effects are presented as standardized β coefficients with the error bars as the 95% CIs. We visualized the absolute effect sizes to aid visual comparison and clarity (see FIGS. 24 and 25 for the raw effect sizes). FIG. 3C, Group differences in DunedinPACNI scores in 1,737 ADNI participants according to cognitive status at scanning. The center lines represent the median. The lower and upper hinges represent the 25th and 75th percentiles. The whiskers extend 1.5 times the interquartile range (IQR) from the hinges. Data beyond the whiskers are plotted as individual outliers. FIG. 3D, Survival curve of the relative proportion of CN ADNI participants at baseline who remained CN during the follow-up window, grouped according to slow, average and fast baseline DunedinPACNI scores. Note that although the maximum follow-up length is 16 years, we chose to visualize only 9 years of follow-up because of high amounts of censoring after 9 years. A plot with the full 16 years of follow-up and points marking censoring is presented in FIG. 11. ADAS-Cog, Alzheimer's Disease Assessment Scale-Cognitive Subscale 13; DSST, Digit Symbol Substitution Task; FAQ, Functional Activities Questionnaire; LogMemory, Logical Memory test; Matrix, Matrix Pattern Completion; MMSE, Mini-Mental State Examination; RAVLT, Rey Auditory Visual Learning Test; Tower, Tower Rearranging; TrailsA, Trail Making Test Part A; TrailsB, Trail Making Test Part B; VM, visual memory; WM, working memory.

FIGS. 4A-4B show DunedinPACNI predicts accelerated hippocampal atrophy. FIG. 4A, Individualized trajectories of hippocampal atrophy in ADNI (left) and UKB (right). Warmer colors represent faster atrophy. FIG. 4B, Forest plot of associations between baseline DunedinPACNI scores and faster hippocampal atrophy in 1,302 ADNI participants and 4,601 UKB participants. Effects are presented as standardized β coefficients with the error bars as the 95% CIs. HC, hippocampus.

FIGS. 5A-5F show DunedinPACNI predicts frailty, poor health, multimorbidity, future chronic diseases and mortality, and reflects social gradients of health inequities. FIG. 5A, Forest plot of absolute associations between DunedinPACNI and frailty (n=42,583) and self-rated health (n=42,235) in UKB. Effects are presented as standardized β coefficients with the error bars as the 95% CIs. FIG. 5B, Group differences in DunedinPACNI scores according to the lifetime number of aging-related chronic disease diagnoses, including myocardial infarction, chronic obstructive pulmonary disease, dementia and stroke in 42,583 UKB participants. FIG. 5C, Survival curve of the relative proportion of disease-free UKB participants at the time of MRI who remained disease-free during the follow-up window, grouped according to slow, average and fast baseline DunedinPACNI scores. We excluded participants who had chronic disease before scanning from this analysis. FIG. 5D, Survival curve of the relative proportion of UKB participants who remained alive during the follow-up window grouped according to baseline DunedinPACNI scores. FIG. 5E, Group differences in DunedinPACNI according to education level in 1,734 ADNI participants. FIG. 5F, Group differences in DunedinPACNI according to education level in 38,297 UKB participants. FIG. 5B, 5E, 5F, The center lines represent the median. The lower and upper hinges represent the 25th and 75th percentiles. The whiskers extend 1.5 times the IQR from the hinges. Data beyond the whiskers are plotted as individual outliers.

FIGS. 6A-6C show DunedinPACNI is similarly associated with dementia and cognitive impairment in a sample of BrainLat participants. FIG. 6A, Group differences in DunedinPACNI scores according to cognitive diagnosis in 369 BrainLat participants. The center lines represent the median. The lower and upper hinges represent the 25th and 75th percentiles. The whiskers extend 1.5 times the IQR from the hinges. Data beyond the whiskers are plotted as individual outliers. FIG. 6B, Forest plot of standardized mean differences in DunedinPACNI between participants with dementia and CN controls in BrainLat (orange; n=369) and ADNI (dark green; n=1,201) while controlling for age and sex. DunedinPACNI was similarly accelerated in dementia in a sample of Latin Americans (BrainLat) and North Americans (ADNI). Effects are presented as standardized β coefficients with the error bars as the 95% CIs. c, Scatter plot of associations between MoCA scores and DunedinPACNI in BrainLat (orange; n=191) and ADNI (dark green, n=1,206) participants. The regression error bands represent the 95% CIs. DunedinPACNI scores were residualized for age and sex. The linear associations between MoCA scores and DunedinPACNI scores were similar in a sample of Latin Americans (BrainLat) and North Americans (ADNI).

FIGS. 7A-7B show a comparison of DunedinPACNI and brain age gap associations with aging-related phenotypes. FIG. 7A, Forest plots of DunedinPACNI and brain age gap absolute effect sizes in ADNI (left) and UKB (right). Effects are presented as standardized β coefficients with the error bars as the 95% CIs. Note that for visualization, the signs of some outcomes were flipped, such that higher scores for all outcomes reflected worse performance or health. Raw effect sizes are presented in FIGS. 24, 25 and Tables 5-7. FIG. 7B, Forest plots of DunedinPACNI and brain age gap HRs in ADNI and UKB. Effects are presented as HRs with the error bars as the 95% CIs. FIG. 7A, 7B, Exact sample sizes for each test are reported in FIGS. 24 and 25 and Tables 5-7). Lighter shades represent the effect size for each measure while controlling for the other measure (that is, the effect of DunedinPACNI when controlling for brain age gap and vice versa). ADAS-Cog, Alzheimer's Disease Assessment Scale-Cognitive Subscale 13; DSST, Digit Symbol Substitution Task; FAQ, Functional Activities Questionnaire; LogMemory, Logical Memory test; Matrix, Matrix Pattern Completion; MMSE, Mini-Mental State Examination; RAVLT, Rey Auditory Visual Learning Test; Tower, Tower Rearranging; TrailsA, Trail Making Test Part A; TrailsB, Trail Making Test Part B; VM, visual memory; WM, working memory.

FIG. 8 shows Dunedin Study attrition analysis using IQ. No significant differences in childhood IQ were found between the full cohort, those still alive, those seen at Phase 45, or those scanned at Phase 45. Those who were deceased by the Phase 45 data collection had significantly lower childhood IQ's than those who were still alive (t=2.09, p=0.04). Center lines of boxes represent the median. Lower and upper hinges of boxes represent the 25th and 75th percentiles. Whiskers show the range. The red line connects the means of each distribution. We report childhood IQ because it is known to be a strong predictor of late-life health outcomes, as shown by many cohort studies from many nations. Childhood IQ predicts health and social outcomes in adulthood, and these outcomes include physical functions, cognitive decline, mental health, inflammation, metabolic syndrome, disease incidence, dementia, mortality, and also neuroimaging-based, genomic, and epigenetic indicators of health. Based on the literature, we report three groups: study members who died before age 45, and thus could not have taken part in data collection, study members who were alive and thus could take part, and study members who actually did take part. We compared these three groups to the original birth cohort. The figure shows that the small group of study members who had died before age 45 had significantly lower mean childhood IQ as a group. Some of the early deaths were Dunedin Study members who had more disadvantages in their lives leading to poorer health and increased risk of early mortality. Study members who died of childhood diseases may have been already unwell at the time of IQ testing, which could have lowered their scores. However, cohort members who are still alive and cohort members who took part in data collection did not differ from the full original cohort on their mean childhood IQ; they still represent population variation on this key health risk factor. Abbreviations: IQ=intelligence quotient.

FIG. 9 shows Dunedin Study attrition analysis using SES. No significant differences were found between the full cohort, those deceased, those alive, those seen at Phase 45, or those scanned at Phase 45 on childhood SES. Center lines of boxes represent the median. Lower and upper hinges of boxes represent the 25th and 75th percentiles. Whiskers show the range. The red line connects the means of each distribution. We report childhood SES because it is known to be a strong predictor of late-life health outcomes, as shown by many cohort studies from many nations. Childhood SES separately predicts health and social outcomes in adulthood, and these outcomes include physical functions, cognitive decline, mental health, inflammation, metabolic syndrome, disease incidence, dementia, mortality, and also neuroimaging-based, genomic, and epigenetic indicators of health. Based on the literature, we report three groups: study members who died before age 45, and thus could not have taken part in data collection, study members who were alive and thus could take part, and study members who actually did take part. We compared these three groups to the original birth cohort. The figure shows that the small group of study members who had died before age 45 had somewhat lower mean childhood SES as a group. Some of the early deaths were Dunedin Study members who had more disadvantages in their lives leading to poorer health and increased risk of early mortality. However, cohort members who are still alive and cohort members who took part in data collection did not differ from the full original cohort on their mean childhood SES; they still represent population variation on this key health risk factor. Abbreviations: SES=socioeconomic status.

FIGS. 10A-10B show associations with DunedinPACNI while excluding participants who go on to have cognitive decline or have high genetic risk for Alzheimer's Disease in the UK Biobank. FIG. 10A. Forest plot of associations between cognitive, frailty, health, and social class measures and DunedinPACNI with and without inclusion of participants who later develop dementia or who carry two APOE E4 risk alleles. Effects are presented as standardized beta coefficients with error bars as 95% confidence intervals. Note that for visualization, the signs of some outcomes were flipped, such that higher scores for all outcomes reflected worse performance or health. FIG. 10B. Forest plot of DunedinPACNI hazard ratios for chronic disease and mortality with and without inclusion of participants who develop dementia or carry two APOE E4 risk alleles. Effects are presented as hazard ratios with error bars as 95% confidence intervals. Exact sample sizes for all tests are presented in Table 2. Abbreviations: DSST=Digit Symbol Substitution Task, HR=hazard ratio, IQ=intelligence quotient, Matrix=Matrix Pattern Completion, RT=Reaction Time, Tower=Tower Rearranging, TrailsA=Trail Making Test Part A, TrailsB=Trail Making Test Part B, VM=Visual Memory, WM=Working Memory.

FIG. 11 shows DunedinPACNI prediction of cognitive decline in ADNI with full follow-up window. Survival curve of the relative proportion of 624 cognitively normal ADNI participants at baseline who remained cognitively normal during the follow-up window grouped by slow, average, and fast baseline DunedinPACNI scores. Censored timepoints (that is, points when participants either converted to MCI/dementia or were lost to follow-up) are shown with cross marks. Note that relatively few participants have >9 years of follow-up. Abbreviations: ADNI=Alzheimer's Disease Neuroimaging Initiative, SD=standard deviation.

FIGS. 12A-12B show cross-sectional cognitive associations with DunedinPACNI, hippocampal volume, and ventricular volume in the UK Biobank. FIG. 12A. Forest plot of associations between cognitive measures and DunedinPACNI and hippocampal volume. Effects are presented as standardized beta coefficients with error bars as 95% confidence intervals. Note that we flipped the direction of hippocampal associations to match the direction of DunedinPACNI. FIG. 12B. Forest plot of associations between cognitive measures and DunedinPACNI and ventricular volume. Effects are presented as standardized beta coefficients with error bars as 95% confidence intervals. Lighter shades represent the effect size for each measure while controlling for the other measure (that is, effect of DunedinPACNI when controlling for hippocampal or ventricular volume, and vice versa). Note that for visualization, the signs of some outcomes were flipped, such that higher scores for all outcomes reflected worse performance or health. Exact effect sizes and sample sizes for each test are presented in FIGS. 27, 28). Abbreviations: DSST=Digit Symbol Substitution Task, Hipp.=hippocampus, IQ=intelligence quotient, Matrix=Matrix Pattern Completion, RT=Reaction Time, Tower=Tower Rearranging, TrailsA=Trail Making Test Part A, TrailsB=Trail Making Test Part B, Vent.=ventricle, vol.=volume, VM=Visual Memory, WM=Working Memory.

FIGS. 13A-13B show cross-sectional frailty and health associations with DunedinPACNI, hippocampal volume, and ventricular volume in the UK Biobank. FIG. 13A. Forest plot of associations between frailty and health measures and DunedinPACNI and hippocampal volume. Effects are presented as standardized beta coefficients with error bars as 95% confidence intervals. Note that we flipped the direction of hippocampal associations to match the direction of DunedinPACNI. FIG. 13B. Forest plot of associations between frailty and health measures and DunedinPACNI and ventricle volume. Effects are presented as standardized beta coefficients with error bars as 95% confidence intervals. Lighter shades represent the effect size for each measure while controlling for the other measure (that is, effect of DunedinPACNI when controlling for hippocampal or ventricular volume, and vice versa). Exact sample sizes for each test are presented in FIGS. 27, 28). Abbreviations: Hipp.=hippocampus, Vent.=ventricle, vol.=volume.

FIGS. 14A-14C show cognitive decline, disease, and mortality prediction by DunedinPACNI, hippocampal volume, and ventricular volume. FIG. 14A. Cognitive decline prediction among cognitively normal ADNI participants, FIG. 14B. Chronic disease prediction in UK Biobank. FIG. 14C. Mortality prediction in UK Biobank. Gray points indicate hazard ratios for each brain measure while covarying for sex and age at scan. The blue point indicates the DunedinPACNI hazard ratio while also covarying for hippocampal volume. The pink point indicates the DunedinPACNI hazard ratio while covarying for ventricular volume. The orange points indicate the hippocampal and ventricular volume hazard ratios while covarying for DunedinPACNI. Note that we flipped the direction of hippocampal associations to match the direction of DunedinPACNI. Error bars on all plots represent 95% confidence intervals. Abbreviations: ADNI=Alzheimer's Disease Neuroimaging Initiative, Hipp.=hippocampus, HR=hazard ratio, UKB=UK Biobank, Vent.=ventricle, vol.=volume.

FIGS. 15A-15B show DunedinPACNI has excellent test-retest reliability. FIG. 15A. Forest plot of intra-class correlations of DunedinPACNI scores calculated in the Human Connectome Project test-retest sample (N=45). Intraclass correlation coefficients are plotted for DunedinPACNI both with and without gray-white signal intensity ratio measures. Error bars represent 95% confidence intervals. FIG. 15B. Correlation between DunedinPACNI estimates at scanning timepoints. Left panel shows DunedinPACNI without gray-white signal intensity ratio measures, right panel shows DunedinPACNI with gray-white signal intensity ratio measures. Both plots include an identity line. Abbreviations: CI=confidence interval, GWR=gray-white signal intensity ratio, ICC=intraclass correlation coefficient.

FIG. 16 shows Dunedin Study member inclusion flowchart. Abbreviations: FLAIR=Fluid attenuated inversion recovery, MRI=magnetic resonance imaging.

FIG. 17 shows ADNI participant inclusion flowchart. Participants were deemed to failed ADNI quality control if they were not given a “Pass” according to centralized quality control of FreeSurfer output by ADNI investigators or if their FreeSurfer output failed visual inspection. Abbreviations: ADNI=Alzheimer's Disease Neuroimaging Initiative, ADSP=Alzheimer's Disease Sequencing Project, CN=cognitively normal, MCI=mild cognitive impairment, QC=quality control.

FIG. 18 shows UK Biobank participant inclusion flowchart. Participants were excluded for low scan signal-to-noise ratio, unusual head positioning, or extremely large intracranial volume or cortical thickness estimates presumed to represent low quality FreeSurfer reconstruction. Abbreviations: CNR=contrast-to-noise ratio, eTIV=estimated total intracranial volume, Head pos=head position, meanCT=mean cortical thickness, MRI=magnetic resonance imaging, SNR=signal-to-noise ratio, UKB=UK Biobank.

FIG. 19 shows BrainLat participant inclusion flowchart. Participants were excluded if their raw T1-weighted scans had incomplete whole-brain coverage or excessive motion corruption, missing demographic data, and diagnoses other than Alzheimer's dementia, frontotemporal dementia, or healthy control. Abbreviations: QC=quality control.

FIGS. 20A-20C show DunedinPACNI is associated with age in ADNI, UK Biobank, and BrainLat participants. Correlation between age and DunedinPACNI in FIG. 20A. ADNI (N=6,204 scans, N=1,737 individuals), FIG. 20B. UK Biobank (N=42,583), and FIG. 20C. BrainLat (N=369). Error bands in all plots represent 95% confidence. ADNI=Alzheimer's Disease Neuroimaging Initiative, CI=confidence interval.

FIGS. 21A-21E show DunedinPACNI is similar with and without gray-white signal intensity ratio phenotypes. DunedinPACNI can still be estimated when gray-white signal intensity ratio measures are not available. FIG. 21A. Correlation between DunedinPACNI estimated with and without gray-white signal intensity ratio measures in the Dunedin Study (N=860). FIG. 21B. Correlation between the Pace of Aging and DunedinPACNI estimated without gray-white signal intensity ratio measures within the Dunedin Study (N=860). Note that this correlation is likely higher than would be expected in an external dataset. Further note that Pace of Aging is standardized to mean=0, SD=1 to facilitate visualization. FIG. 21C. Correlation between DunedinPACNI estimated with and without gray-white signal intensity ratio in the ADNI cross-sectional sample (N=6,204 scans, N=1,737 individuals). FIG. 21D. Correlation between DunedinPACNI estimated with and without gray-white signal intensity ratio in the UK Biobank sample (N=42,583). FIG. 21E. Correlation between DunedinPACNI estimated with and without gray-white signal intensity ratio in the BrainLat sample (N=369). Error bands in all plots represent 95% confidence. Abbreviations: ADNI=Alzheimer's Disease Neuroimaging Initiative, CI=confidence interval, GWR=gray-white signal intensity ratio.

FIG. 22 shows an example process in accordance with some embodiments of the methods disclosed herein.

FIG. 23 shows an example system in accordance with some embodiments of the systems disclosed herein.

FIG. 24 shows a table detailing associations between DudenPACNI, brain age gap, and cognition in ADNI.

FIG. 25 shows a table detailing associations between DudenPACNI, brain age, and cognition in the UK Biobank.

FIG. 26 shows a table detailing Associations between DunedinPACNI, hippocampal volume, and outcomes in UK Biobank participants.

FIG. 27 shows a table detailing associations between DunedinPACNI, ventricular volume, and outcomes in UK Biobank participants.

FIG. 28 shows a table detailing associations between DunedinPACNI, hippocampal volume, ventricular volume, and risk for cognitive decline, chronic disease, and death

### DETAILED DESCRIPTION

Provided herein are systems and methods for estimating an aging metric of a subject. Aging affects functional decline and increases disease risk. However, it has been shown that individuals can age at different speeds. Individuals who age at a faster rate than average may be at an increased risk of developing a disorder.

In the Examples, it is demonstrated that neurological imaging can be used to estimate a subject's pace of aging and assess the likelihood of developing a disorder based on the pace of aging. This can be quantified using several approaches outlined in the Examples.

In one aspect, a method of estimating a metric of health of a subject is disclosed. The method may include acquiring brain imaging of the subject; extracting a plurality of structural measures from the brain imaging data; applying a trained model to the plurality of structural measures to determine a pace of aging score; comparing the pace of aging score of the subject to a normative reference distribution to determine a centile score, in which the normative reference includes a plurality of pace of aging scores corresponding to a plurality of control subjects; and outputting the metric of health, in which the metric of health includes the pace of aging score and the centile score of the subject.

As used herein, a “subject” may be interchangeable with “patient” or “individual” and means a human in need of treatment. A “subject in need of treatment” may include a subject who has been identified as being likely to have or to develop a disorder or condition that is responsive to a therapy.

### Brain Imaging

The systems and methods described herein use brain-based measures to directly estimate longitudinal aging of a subject. Therefore, brain imaging data (e.g., neural imaging data) is used as an input to the systems and methods. The brain imaging must be able to elucidate a plurality of structural measures in the subject's brain.

Magnetic resonance imaging (MRI) data may be used as brain imaging data. In some embodiments, T1-weighted MRI scans may be used as brain imaging data.

In some embodiments, the plurality of structural measures observed in the subject's brain is over 300. In some embodiments, 315 structural measures are used to derive pace of aging scores. The plurality of structural measures may be limited to the plurality of structural measures comprising at least one of regional cortical thickness, surface area, gray matter volume, gray-white matter signal intensity ratio, subcortical thickness, ventricular volumes, or bilateral volume of white matter hypointensities.

### Estimating Metrics of Aging

Several measures have been developed to index biological aging. Biomarkers of biological age and accelerated biological aging are correlated with disease, predict future disease risk, and are used as primary or secondary outcomes in clinical trials of preventative interventions. However, it is challenging to interpret these biomarkers without age- and sex-based reference norms. In other words, biomarker scores can be compared to one another using regression-based analyses, but these scores cannot be used to independently measure and interpret any single patient's risk. In pediatrics, normative growth charts are used to assess the degree to which a child's height and weight deviate from other children of the same age and sex (i.e., the child's percentile rank). Constructing analogous norms for aging biomarkers would allow clinicians to identify whether a patient is aging more rapidly than what is normal for their age and sex, thereby facilitating interpretation and informing treatment.

Developing normative reference charts for DunedinPACNI is particularly timely as the Alzheimer's Association multi-disciplinary Diagnostic Evaluation, Testing, Counseling, and Disclosure of Suspected Alzheimer's Disease and Related Disorders (DETeCD-ADRD) expert workgroup introduced in 2025 new evidence-based guidelines for screening and diagnosis of suspected dementia that includes a T1-weighted brain MRI as a first tier test in both primary care settings and specialty clinics. The inclusion of brain MRI whenever possible in suspected cases of dementia presents a clear opportunity to measure DunedinPACNI within existing clinical workflows at little additional cost. The normative reference charts presented herein quantify the relative dementia risk associated with accelerated whole-body aging and could help communicate this risk to clinicians and patients.

The methods and systems disclosed herein may include using a trained model to determine a metric of aging (e.g., a pace of aging score or a centile score) of a subject. In some embodiments, the trained model may be trained on longitudinal data from a plurality of subjects, in which the longitudinal data comprises brain imaging data and information on at least one disease state collected at a plurality of time points. The subjects included in the longitudinal data may include control subjects who have had brain imaging data collected over their lifespans (e.g., from 18-80 or 100 years old). In some embodiments, the control subjects may be subjects who have not been diagnosed with a psychiatric disorder.

The plurality of time points may be set times or ages to collect new brain imaging. For instance, a control subject may have brain imaging data collected every five years, ten years, fifteen years, or twenty years. Alternatively, the control subject may have brain imaging data collected at specific ages (e.g., 30 years old, 40 years old, 50 years old, etc.).

The model may be trained using a regression model. The regression model may include at least one of a linear regression, logistic regression, polynomial regression, ridge regression, lasso regression, quantile regression, or elastic net regression. In some embodiments, the regression model includes an elastic net regression.

Different models may be used to provide a close match to a specific subject. For instance, a model may be trained using data with one sex. Therefore, a model may be selected based on the sex of a subject. If a subject's sex is female, a model that was trained exclusively on control subjects that have a female sex.

Quantification of accelerated whole-body aging through DunedinPACNI centile scores can benefit risk assessment for other chronic age-related diseases. DunedinPACNI likewise could provide an outcome measure in clinical trials of anti-aging therapeutics.

The Examples demonstrate how normative reference charts were used to determine whether a person's pace of aging is accelerated beyond what is typical for their age and sex. Specifically, centile scores may be calculated. Centile scores indicate the percentile of people of the same age and sex that are below a certain person's score (e.g., a person with a centile score of 86 would have a DunedinPACNI score above 86% of people of the same age and sex).

The “same” age as a subject may refer to an age within 12-months of the subject. The same “sex” as a subject may refer to the sex assigned at birth.

### Longitudinal Monitoring

The systems and methods described herein may be used for longitudinal monitoring of a subject's aging. The longitudinal monitoring may include: obtaining a second set of brain imaging of the subject at a later point in time; determining a corresponding updated centile score of the subject using the method of any one of the preceding claims; quantifying a change in the centile score; and outputting the second metric of health and the change in centile score. The second set of brain imaging of the subject may be collected at least 1 year, at least 2 years, at least 3 years, at least 4 years, at least 5 years, at least 6 years, at least 7 years, at least 8 years, at least 9 years or at least 10 years after brain imaging data was originally collected. Longitudinal monitoring is not limited to two time points; brain imaging data may be collected at set frequencies, and changes to metrics of aging may be determined. These changes may be used to determine the likelihood of a subject developing a disease or condition and also to prescribe appropriate interventions and/or monitor whether prescribed interventions are effective to delay onset or progression of a disease or condition.

### Predicting Likelihood of Developing a Disorder

The systems and methods described herein may be used to predict a subject's likelihood of developing a disorder. A subject's centile score may be used to determine the subject's likelihood of developing a disorder. For instance, it may be determined if a subject's centile score is “higher” than control subjects that are controlled for sex and age. In some embodiments, if the centile score is above 50 (e.g., at least 50% of control subjects have a pace of aging below the subject's), then the subject is at higher risk for developing a chronic disease or condition.

In some embodiments, the systems and methods disclosed herein may be used to determine the likelihood that a subject develops a chronic disorder. The chronic disorder may be selected from the group including stroke, chronic obstructive pulmonary disease, myocardial infarct, and dementia. In some embodiments, the chronic disorder may be a neurological disorder such as dementia.

The Examples demonstrate that DunedinPACNI predicts risk for dementia independently of the leading established factors that are now used by clinicians to estimate a patient's risk of developing dementia. People with DunedinPACNI scores above 50th percentile were 2.0 times more likely to develop dementia. People at low genetic risk for Alzheimer's disease (APOE &4 noncarriers) with DunedinPACNI scores above 50th percentile were 2.5 times more likely to develop dementia. People at high genetic risk for Alzheimer's disease (APOE &4 heterozygotes) with DunedinPACNI scores above 50th percentile were 1.7 times more likely to develop dementia. People with healthy brains (normal hippocampal volume) with DunedinPACNI scores above 50th percentile were 2.2 times more likely to get dementia. People with unhealthy brains (abnormal hippocampal volume) with DunedinPACNI scores above 75th percentile were 2.2 times more likely to get dementia.

In some embodiments, the chronic disorder may refer to general health or mortality.

The Examples demonstrate that DunedinPACNI predicts mortality and does so regardless of how many chronic diseases a person has. Specifically: People who had DunedinPACNI scores above 50th percentile were at 1.4 times higher risk of death from any cause. People without any chronic diseases who had DunedinPACNI scores above 50th percentile were at 1.3 times higher risk of death from any cause. People who had one chronic disease and had DunedinPACNI scores above 50th percentile were at 1.3 times higher risk of death from any cause. People who had two or more chronic diseases and had DunedinPACNI scores above 50th percentile were at 2.4 times higher risk of death from any cause. Altogether, this means that DunedinPACNI predicts risk for dementia and mortality independently of existing risk factors.

### Methods of Treatment

The systems and methods described herein may be used to administer a recommended intervention to the subject. A recommended intervention may be administered to mitigate the risk of developing a disorder or delay onset or progression of a disorder. The recommended intervention may include non-medical interventions, or medical interventions.

Non-medical interventions include, but are not limited to, a change in lifestyle, behavioral therapy, or some combination thereof. A change in lifestyle may include at least one of limiting tobacco intake, limiting alcohol intake, modifying diet, increasing physical activity, or obtaining a healthy weight. Each of these lifestyle changes has been shown to decrease the likelihood of developing more than one chronic disorder.

Modifying diet may specifically refer to adopting a low-fat diet, a Mediterranean diet (MeDi diet), a Dietary Approach to Stop Hypertension (DASH), or some combination thereof. Increasing physical activity may include increasing the frequency a subject engaged in aerobic exercise programs, resistance exercise programs, flexibility and balance exercise programs, or mixed exercise programs. Behavioral therapy may include at least one of at least one of talk therapy, cognitive-behavioral therapy, or behavioral activation therapy.

In some embodiments, longitudinal monitoring may be used to update recommended interventions. For instance, if a subject's centile score increases during longitudinal monitoring, a new intervention may be recommended. A new intervention may be added (e.g., a medical intervention is recommended in addition to a non-medical intervention), or an existing intervention may be administered more strictly (e.g., stricter adherence to a recommended diet).

A recommended intervention may be individualized based on the specific disorder a subject is likely to develop. For instance, a recommended intervention may include administering an additional therapy and/or a supplement.

In cases in which a disorder is likely to be stroke, chronic obstructive pulmonary disease, or myocardial infarct, the recommended intervention further comprises intake of a supplement, in which the supplement includes magnesium, folic acid, CoQ10, phytosterols, L-carnitine, vitamin B, or some combination thereof.

In cases in which a disorder is likely to be dementia, the recommended intervention may include administering cognitive therapy or an intervention targeting cardiovascular pathology. The therapy may include activities to improve at least one of memory, reasoning, processing speed, executive functioning, problem solving, or general knowledge. The recommended therapy may include administration of supplements known to have positive impacts on cognition, such as at least one of vitamins, minerals, antioxidants, vitamin A, vitamin B, vitamin C, vitamin D, vitamin E, vitamin docosahexaenoic acid (DHA), magnesium, selenium, chromium, zinc, carotenoids, flavonoids, polyphenols, omega-3, or some combination thereof.

The recommended intervention may include a medical intervention. A medical intervention may include an appointment with a medical professional to determine a best course of action or a prescription medication. The medical intervention may further include routine appointments to monitor the potential onset of a disorder. The medical intervention may further include administering a drug or pharmaceutical compound.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference unless explicitly indicated otherwise. The present disclosure shall control in the event there are any disparities between any definitions and/or descriptions found in the cited references.

The following examples are meant only to be illustrative and are not meant as limitations on the scope of the invention or of the appended claims.

### EXAMPLES

### Overview

To understand how aging affects functional decline and increases disease risk, it is necessary to develop measures of how fast a person is aging. Using data from the Dunedin Study, we introduce an accurate and reliable measure for the rate of longitudinal aging derived from cross-sectional brain magnetic resonance imaging, that is, the Dunedin Pace of Aging Calculated from NeuroImaging (DunedinPACNI). Exporting this measure to the Alzheimer's Disease Neuroimaging Initiative, UK Biobank and BrainLat datasets revealed that faster DunedinPACNI predicted cognitive impairment, accelerated brain atrophy and conversion to diagnosed dementia. Faster DunedinPACNI also predicted physical frailty, poor health, future chronic diseases and mortality in older adults. DunedinPACNI is a next-generation brain magnetic resonance imaging biomarker that can help researchers explore aging effects on health outcomes and evaluate the effectiveness of antiaging strategies.

### Introduction

Aging is the gradual, progressive and correlated decline of multiple organ systems over decades. Longitudinal studies provide evidence for substantial individual variation in the rate of aging; people born in the same year can age slower or faster than their peers1-3. Furthermore, aging itself is increasingly regarded as a potentially preventable cause of chronic disease. Accordingly, accurate and reliable measures of how fast a person is aging are needed to effectively study how individual variation in the rate of aging contributes to disease risk and to evaluate interventions intended to slow aging before irreversible decline4-8.

Age-sensitive alterations in DNA methylation, referred to as epigenetic clocks, are currently the most widely used measures for estimating individual differences in aging4,9,10. First-generation epigenetic clocks were trained on chronological age11,12, but the more precisely they predicted chronological age, the less well they predicted clinical outcomes 13,14. In response, second-generation clocks were trained on measures of health that predict mortality in older people15-17. However, these clocks were trained on cross-sectional phenotypes in multiage samples, not on longitudinal observations of the same person as recommended in geroscience5,18. This limitation led to the development of a third-generation longitudinal approach to measuring aging.

We previously adopted this longitudinal approach in the Dunedin Study, which has followed a population representative sample of 1,037 people born in the same year (1972-1973) from birth to age 45 (ref. 19). Across two decades (ages=26, 32, 38 and 45 years), we repeatedly measured 19 biomarkers of cardiovascular, metabolic, renal, immune, dental and pulmonary functioning. By averaging the decline in the trajectories of these biomarkers, we operationalized the theoretical construct of biological aging into a specific measure that we called the Pace of Aging2. We subsequently developed an epigenetic clock that accurately and reliably estimates the Pace of Aging: the Dunedin Pace of Aging Calculated from the Epigenome (DunedinPACE) 20. Because DunedinPACE is calculated from a single time point measurement of DNA methylation, it has been rapidly adopted by studies on aging, where it has been associated with signs of accelerated brain aging, morbidity and mortality20-25. However, it has not been possible to export DunedinPACE or other epigenetic clocks to studies lacking DNA methylation data. This includes many neuroimaging studies of brain aging and neurodegenerative diseases such as Alzheimer's disease (AD).

Current neuroimaging-based approaches to measure aging, akin to first-generation epigenetic clocks, involve training models to predict chronological age from variability in magnetic resonance imaging (MRI) measures of brain structure in large multiage samples26-30. As with first-generation epigenetic clocks, these age deviation approaches unavoidably mix model error (for example, historical differences in environmental exposures, survivor bias, disease effects, measurement bias) with a person's true rate of biological aging31-33.

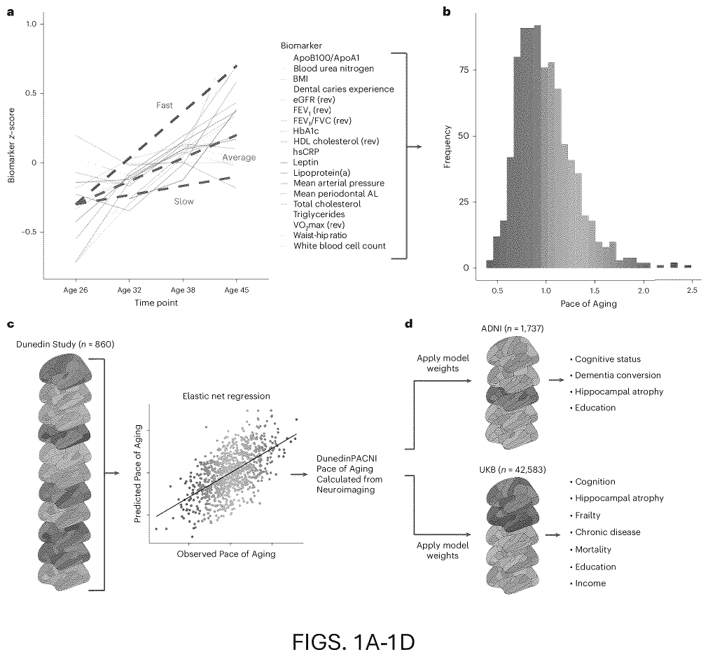

In this study, using a single T1-weighted MRI scan collected at age 45 in the Dunedin Study, we describe the development and validation of a brain MRI measure for the Pace of Aging (FIGS. 1A-1C). We call this measure Dunedin Pace of Aging Calculated from NeuroImaging (DunedinPACNI). Using data from the Human Connectome Project (HCP), we evaluated the test-retest reliability of DunedinPACNI. Exporting the measure to the Alzheimer's Disease Neuroimaging Initiative (ADNI), UK Biobank (UKB) and Latin American Brain Health Institute (BrainLat) datasets, we conducted a series of preregistered analyses designed to evaluate the utility of DunedinPACNI for predicting multiple aging-related health outcomes (FIG. 1D). To benchmark our findings, we compared the effect sizes for DunedinPACNI to those for brain age gap34. DunedinPACNI is a brain-based measure trained to directly estimate longitudinal aging of non-brain organ systems. Therefore, if DunedinPACNI indeed estimates individual differences in the rate of aging, it would add evidence for close links between brain integrity and whole-body aging and establish neuroimaging as a powerful tool for measuring aging, that is, not just of the brain but of the entire body35.

### Results

### DunedinPACNI: A Brain MRI Measure of Longitudinal Aging

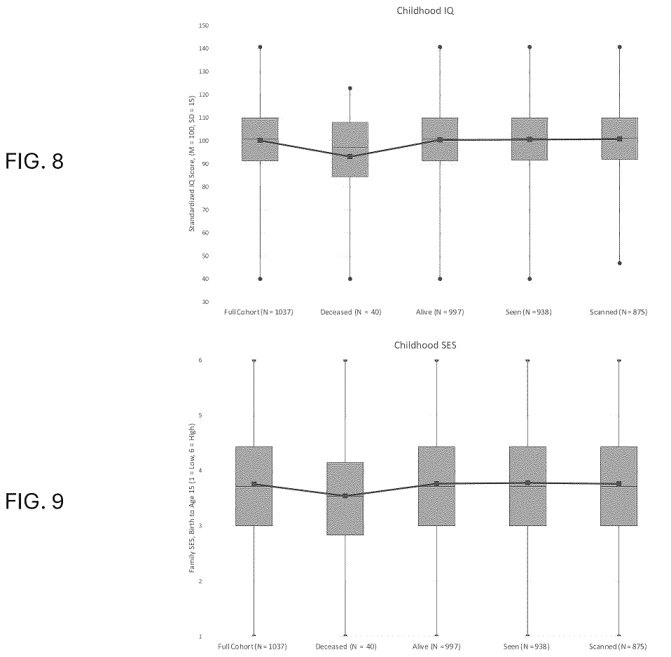

We trained an elastic net regression model to predict the longitudinal Pace of Aging measure using T1-weighted MRI scans collected in a subsample of 860 Dunedin Study members when they were 45 years old. This subsample maintains the population representativeness of the full cohort (FIG. 8, FIG. 9). Specifically, the elastic net regression model used 315 MRI-derived structural measures for each study member, including regional cortical thickness (CT), surface area (SA), gray matter volume (GMV) and gray-white signal intensity ratio (GWR), as well as subcortical gray matter and ventricular volumes36. We performed tenfold cross-validation to identify the optimal tuning parameters37. This optimized model was used to create DunedinPACNI.

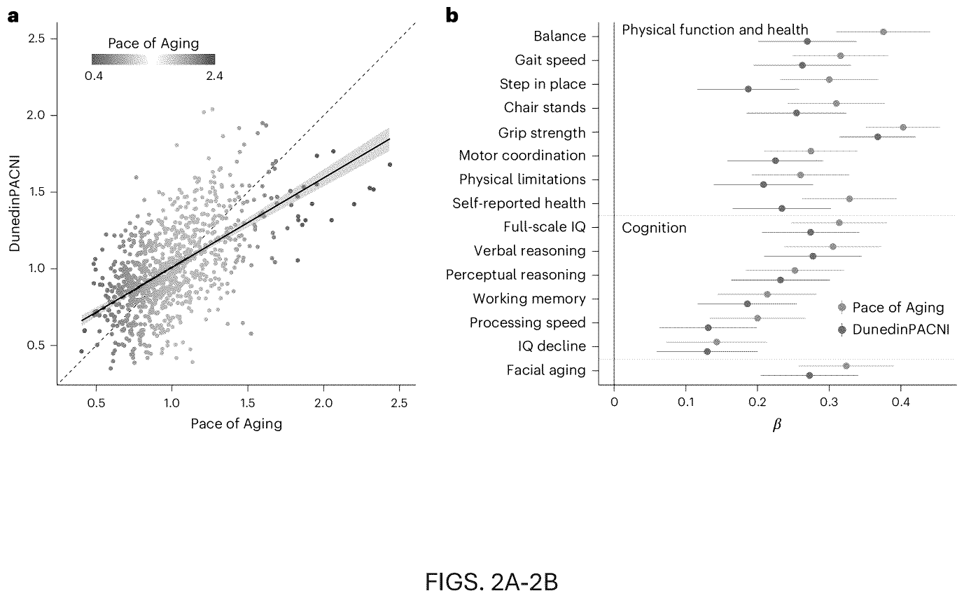

The in-sample correlation between DunedinPACNI and the longitudinal Pace of Aging was r=0.60 (FIG. 2A). We performed a cross-validation analysis by splitting the sample into training and testing subsets 100 different times. Each time, we used 90% of the sample for training and held out the remaining 10% for testing. Across all 100 different splits, the average correlation between DunedinPACNI and Pace of Aging in the testing sample was r=0.42. This prediction accuracy is in line with next-generation epigenetic biomarkers of aging15,20,38. For both DunedinPACNI and the longitudinal Pace of Aging, higher scores indicate faster aging. Note that DunedinPACNI is a quantitative variable that indexes relative differences between people without clear units. For this reason, we restricted our interpretation to directional comparisons within studies. Associations between faster DunedinPACNI scores and measures of physical functioning, cognitive functioning and facial aging were similar to those previously observed with Pace of Aging2. In these analyses, we controlled for sex but not age because all Dunedin Study members have the same chronological age. The DunedinPACNI effect sizes for 12 of the 15 measures were within the 95% confidence intervals (CIs) of the original Pace of Aging (FIG. 2B; Table 1). This was expected given the high internal correlation between DunedinPACNI and Pace of Aging. Dunedin Study members with faster DunedinPACNI scores had worse balance, slower gait, weaker lower-body and upper-body strength, and poorer coordination; they also reported worse health and more physical limitations; performed more 5 poorly on tests of cognitive functioning; experienced greater childhood-to-adulthood cognitive decline; and looked older. These results indicate that DunedinPACNI accurately estimates the longitudinal Pace of Aging in the Dunedin Study dataset.

| | | | | 95% CI | 95% CI |
|---|---|---|---|---|---|
| Phenotype | Measure | beta | p-value | lower bound | upper bound |
| Balance | DunedinPACNI | −0.26 | <0.0001 | −0.33 | −0.19 |
| | Pace of Aging | −0.36 | <0.0001 | −0.43 | −0.30 |
| Gait speed | DunedinPACNI | −0.26 | <0.0001 | −0.32 | −0.19 |
| | Pace of Aging | −0.32 | <0.0001 | −0.38 | −0.26 |
| Step in place | DunedinPAONI | −0.19 | <0.0001 | −0.26 | −0.12 |
| | Pace of Aging | −0.31 | <0.0001 | −0.37 | −0.24 |
| Chair Stands | DunedinPACNI | −0.23 | <0.0001 | −0.30 | −0.17 |
| | Pace of Aging | −0.31 | <0.0001 | −0.37 | −0.24 |
| Grip strength | DunedinPACNI | −0.36 | <0.0001 | −0.41 | −0.31 |
| | Pace of Aging | −0.39 | <0.0001 | −0.44 | −0.34 |
| Motor coordination | DunedinPACNI | 0.24 | <0.0001 | 0.17 | 0.30 |
| | Pace of Aging | 0.26 | <0.0001 | 0.19 | 0.32 |
| Self-reported physical | DunedinPACNI | 0.24 | <0.0001 | 0.17 | 0.30 |
| limitations | Pace of Aging | 0.28 | <0.0001 | 0.21 | 0.34 |
| Self-rated health | DunedinPAGNI | −0.25 | <0.0001 | −0.31 | −0.18 |
| | Pace of Aging | −0.36 | <0.0001 | −0.42 | −0.29 |
| Full-scale IQ | DunedinPACNI | −0.27 | <0.0001 | −0.34 | −0.21 |
| | Pace of Aging | −0.31 | <0.0001 | −0.38 | −0.25 |
| Verbal reasoning | DunedinPACNI | −0.26 | <0.0001 | −0.33 | −0.20 |
| | Pace of Aging | −0.30 | <0.0001 | −0.36 | −0.23 |
| Perceptual reasoning | DunedinPACNI | −0.25 | <0.0001 | −0.31 | −0.18 |
| | Pace of Aging | −0.25 | <0.0001 | −0.32 | −0.19 |
| Working memory | DunedinPACNI | −0.17 | <0.0001 | −0.24 | +0.11 |
| | Pace of Aging | −0.20 | <0.0001 | −0.27 | −0.14 |
| Processing speed | DunedinPACNI | −0.15 | <0.0001 | −0.21 | −0.08 |
| | Pace of Aging | −0.22 | <0.0001 | −0.28 | −0.15 |
| IQ decline | DunedinPACNI | −0.12 | 0.0003 | −0.19 | −0.06 |
| | Pace of Aging | −0.15 | <0.0001 | −0.22 | −0.09 |
| Facial aging | DunedinPACNI | 0.26 | <0.0001 | 0.19 | 0.32 |
| | Pace of Aging | 0.33 | <0.0001 | 0.26 | 0.39 |

Abbreviations:
CI = confidence interval,
IQ = intelligence quotient.

Table 1: Associations between DunedinPACNI and cognition, physical function, and subjective aging in the Dunedin Study.

### DunedinPACNI has Excellent Test-Retest Reliability

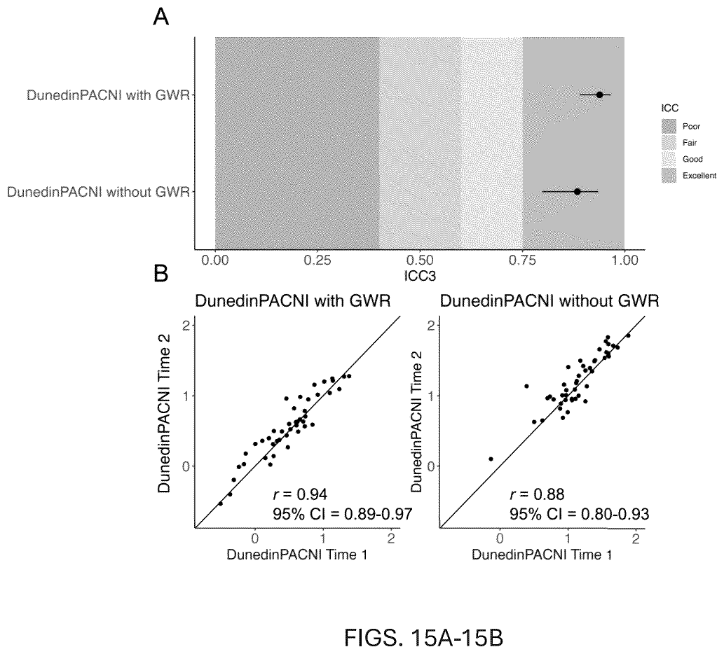

If DunedinPACNI is to be used as a measure of aging, it must exhibit sufficient measurement reliability when exported to new datasets. We used test-retest MRI data (n=45) from the HCP49 to estimate the reliability of DunedinPACNI. The test-retest reliability was excellent (intraclass correlation coefficient=0.94, 95% CI=0.89 to 0.97; FIGS. 15A-15B).

DunedinPACNI is Associated with Worse Cognitive Functioning

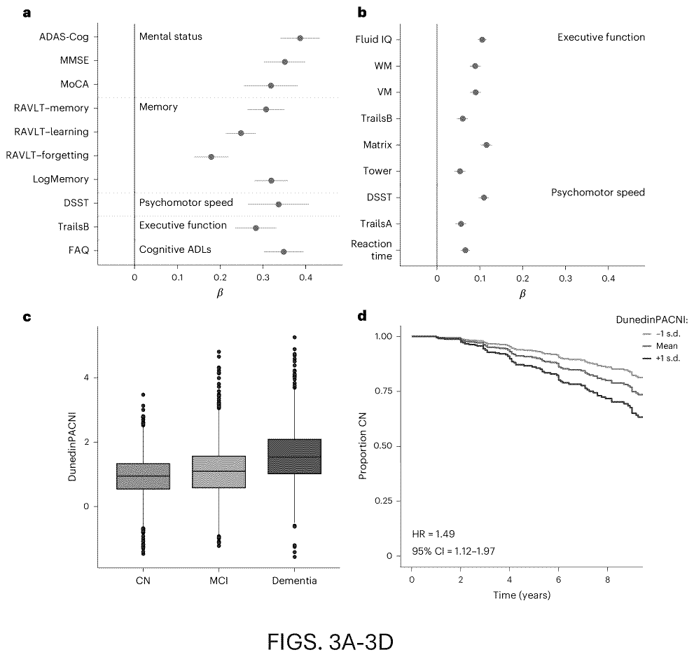

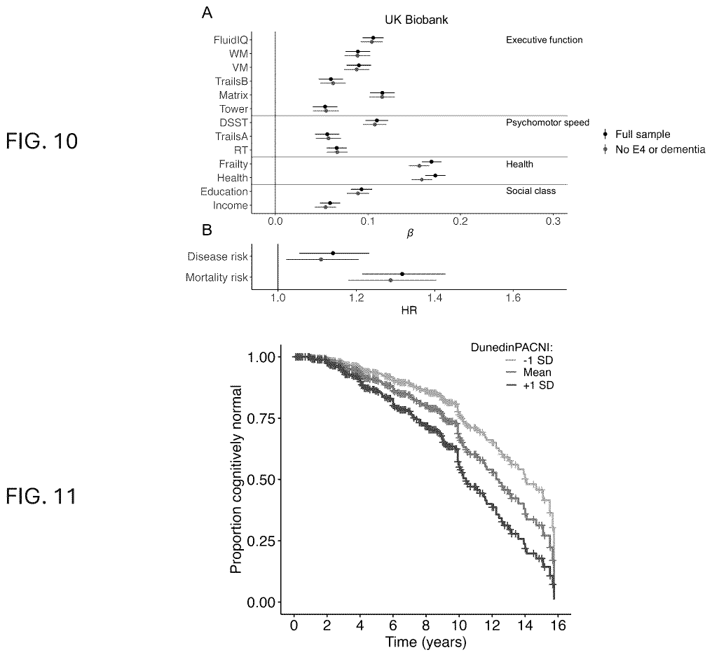

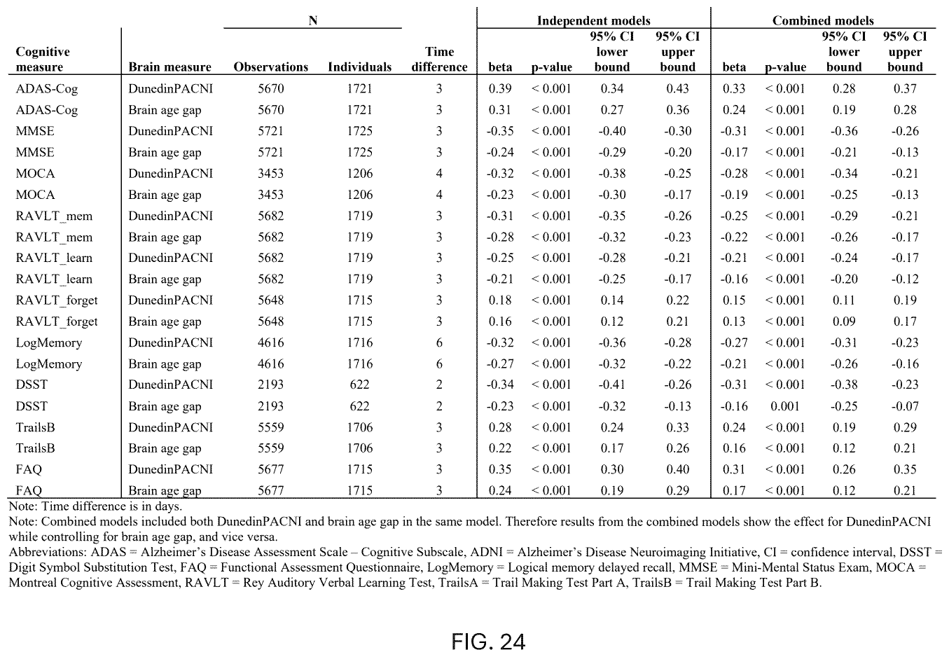

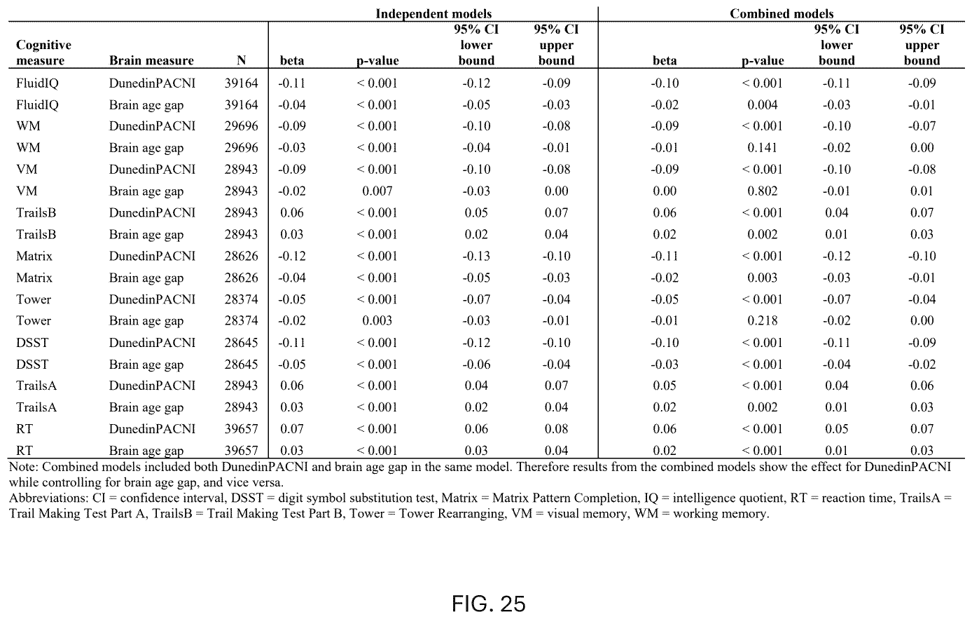

Having established both internal validity and test-retest reliability, we sought to examine whether DunedinPACNI generalizes to new datasets to detect aging-related outcomes. Specifically, we first tested for associations with cognitive impairment in ADNI and cognitive functioning in UKB. We generated DunedinPACNI scores from T1-weighted MRI scans collected in 1,737 ADNI participants (mean age=74.3 years, s.d.=7.2, range=52-97 years) and 42,583 UKB participants (mean age=64.4 years, s.d.=12.7, range=44-82 years). In ADNI, participants with faster DunedinPACNI showed greater impairment on mental status examinations used to screen for dementia, as well as tests of memory, psychomotor speed and executive functioning. They also reported more impairment in cognitively demanding activities of daily living (ADLs), such as maintaining finances or preparing a meal (FIG. 3A). Absolute standardized effect sizes across all cognitive measures in ADNI ranged from β=0.18 to 0.39 (all P<0.001; full results in FIG. 24). Similarly, UKB participants with faster DunedinPACNI performed more poorly on tests of executive functioning and psychomotor speed (FIG. 3B). Absolute standardized effect sizes across all cognitive measures in UKB ranged from β=0.05 to 0.12 (all P<0.001; full results in FIG. 25). Associations in UKB were not driven by individuals with early cognitive decline or APOE &4 homozygotes, who have heightened genetic risk for Alzheimer's disease and show earlier onset of cognitive decline50 (FIG. 10 and Table 2). These, and all subsequent analyses presented in this article, control for sex and chronological age.

| | Full sample | | | | | No double APOE E4 carriers or later dementia | | | | |
|---|---|---|---|---|---|---|---|---|---|---|
| | | | | 95% CI | 95% CI | | | | 95% CI | 95% CI |
| | | | | lower | upper | | | | lower | upper |
| Outcome | N | beta | p-value | bound | bound | N | beta | p-value | bound | bound |
| FluidIQ | 39164 | −0.11 | <0.001 | −0.12 | −0.09 | 36236 | −0.10 | <0.001 | −0.12 | −0.09 |
| WM | 29696 | −0.09 | <0.001 | −0.30 | −0.08 | 27545 | −0.09 | <0.001 | −0.10 | −0.07 |
| VM | 28943 | −0.09 | <0.001 | −0.10 | −0.08 | 26863 | −0.09 | <0.001 | −0.10 | −0.07 |
| TrailsB | 28943 | 0.06 | <0.001 | 0.05 | 0.07 | 26863 | 0.06 | <0.001 | 0.05 | 0.08 |
| Matrix | 28626 | −0.12 | <0.001 | −0.13 | −0.10 | 26584 | −0.12 | <0.001 | −0.13 | −0.10 |
| Tower | 28374 | −0.05 | <0.001 | −0.07 | −0.04 | 26352 | −0.05 | <0.001 | −0.07 | −0.04 |
| DSST | 28645 | −0.11 | <0.001 | −0.12 | −0.10 | 26596 | −0.11 | <0.001 | −0.12 | −0.09 |
| TrailsA | 28943 | 0.06 | <0.001 | 0.04 | 0.07 | 26863 | 0.06 | <0.001 | 0.04 | 0.07 |
| RT | 39657 | 0.07 | <0.001 | 0.06 | 0.08 | 36678 | 0.07 | <0.001 | 0.06 | 0.08 |
| Frailty | 42583 | 0.17 | <0.001 | 0.16 | 0.18 | 39337 | 0.16 | <0.001 | 0.14 | 0.17 |
| Health | 42235 | −0.17 | <0.001 | −0.18 | −0.16 | 39029 | −0.16 | <0.001 | −0.17 | −0.15 |
| | Full sample | | | | | No double APOE E4 carriers or later dementia | | | | |
| | | | | 95% CI | 95% CI | | | | 95% CI | 95% CI |
| | | | | lower | upper | | | | lower | upper |
| Outcome | N | HR | p-value | bound | bound | N | HR | p-value | bound | bound |
| Chronic | 40753 | 1.14 | 0.001 | 1.05 | 1.23 | 37696 | 1.13 | 0.014 | 1.02 | 1.21 |
| disease | | | | | | | | | | |
| Mortality | 42583 | 1.32 | <0.001 | 1.22 | 1.43 | 39337 | 1.29 | <0.001 | 1.18 | 1.40 |

Table 2: Associations with DunedinPACNI while excluding participants who go on to have cognitive decline or have high genetic risk for Alzheimer's Disease in the UK Biobank. Abbreviations: CI=confidence interval, DSST=Digit Symbol Substitution Task, HR=hazard ratio, IQ=intelligence quotient, Matrix=Matrix Pattern Completion, RT=Reaction Time, Tower=Tower Rearranging, TrailsA=Trail Making Test Part A, TrailsB=Trail Making Test Part B, VM=Visual Memory, WM=Working Memory.

### DunedinPACNI Predicts Future Cognitive Decline and Dementia

We next tested if DunedinPACNI differentiates between normal and clinically impaired cognitive functioning in ADNI (FIG. 3C). Participants with mild cognitive impairment (MCI) had faster DunedinPACNI compared to cognitively normal (CN) participants (β=0.27, P<0.001, 95% CI=0.18 to 0.35). Participants with dementia had faster DunedinPACNI than both participants with MCI (β=0.54, P<0.001, 95% CI=0.43 to 0.65) and CN participants (β=0.81, P<0.001, 95% CI=0.69 to 0.92).

We further tested whether DunedinPACNI predicts future cognitive decline in people without cognitive impairment. Specifically, we analyzed a subsample of 624 ADNI participants who were CN at the time of their first scan (mean age=72.4 years, s.d.=6.3 years, range=52.7-89.9 years), 112 of whom converted to either MCI or dementia during up to 16 years of follow-up (mean follow-up=4.90 years). CN participants with faster DunedinPACNI at baseline were more likely to develop MCI or dementia and to do so earlier during the follow-up window (hazard ratio (HR)=1.49, P=0.005, 95% CI=1.12 to 1.97; FIG. 3D), meaning that those in the top 10% had a 61% increased risk of developing MCI or dementia compared to participants with an average DunedinPACNI. We conducted a similar analysis in the 701 participants who were diagnosed with MCI at the time of their first scan (mean age=72.8 years, s.d.=7.3 years, range=55.0-88.8 years), 271 of whom converted to dementia during up to 16 years of follow-up (mean follow-up=4.10 years). Participants with MCI with faster DunedinPACNI at baseline were more likely to convert to dementia (HR=1.44, P<0.001, 95% CI=1.26 to 1.65). These effect sizes were similar when controlling for the number of APOE 24 alleles, a well-established genetic risk allele for sporadic, late-onset AD (baseline CN: HR=1.49, P=0.005, 95% CI=1.13 to 1.96; baseline MCI: HR=1.42, P<0.001, 95% CI=1.23 to 1.62). Because only a very small number of UKB participants with MRI data received diagnoses of dementia during the follow-up observation (n=73), we were underpowered to report parallel results in this dataset.

### DunedinPACNI Predicts Accelerated Brain Atrophy

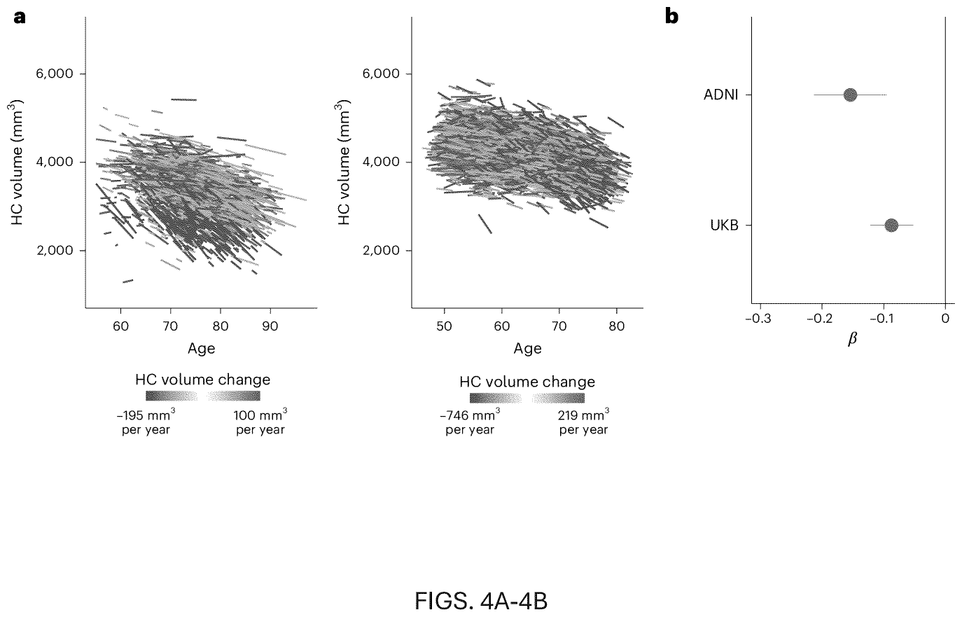

As an estimate of how fast a person is aging, DunedinPACNI should reflect longitudinal trajectories of brain decline33. We tested whether faster baseline DunedinPACNI predicted accelerated hippocampal atrophy, which is an established risk factor for cognitive decline and dementia onset in older adults51. Specifically, we computed longitudinal trajectories of hippocampal atrophy among 1,302 ADNI participants who had MRI data at multiple time points (average number of scans=4.4, range=2-13 scans) as well as 4,601 UKB participants who had MRI data at two time points (FIG. 4A). Participants with faster baseline DunedinPACNI exhibited accelerated hippocampal atrophy in both ADNI (β=−0.15, P<0.001, 95% CI=−0.21 to −0.10; FIG. 4B) and UKB (β=−0.09, P<0.001, 95% CI=−0.12 to −0.05; FIG. 4B). This result was consistent while controlling for number of APOE ε4 alleles (Table 3).

| | | Controlling for APOE E4 genotype | | | | Original model | | | |
|---|---|---|---|---|---|---|---|---|---|
| | | | | 95% CI | 95% CI | | | 95% CI | 95% CI |
| | | | | lower | upper | | | lower | upper |
| Dataset | N | beta | p-value | bound | bound | beta | p-value | bound | bound |
| ADNI | 1302 | −0.14 | <0.001 | −0.20 | −0.08 | −0.15 | <0.001 | −0.21 | −0.10 |
| UK Biobank | 4601 | −0.09 | <0.001 | −0.12 | −0.06 | −0.09 | <0.001 | −0.12 | −0.05 |

Abbreviations:
ADNI = Alzheimer's Disease Neuroimaging Initiative,
CI = confidence interval.

Table 3: Associations between DunedinPACNI and hippocampal atrophy while controlling for APOE E4 genotype.

### DunedinPACNI Predicts Frailty, Disease and Mortality

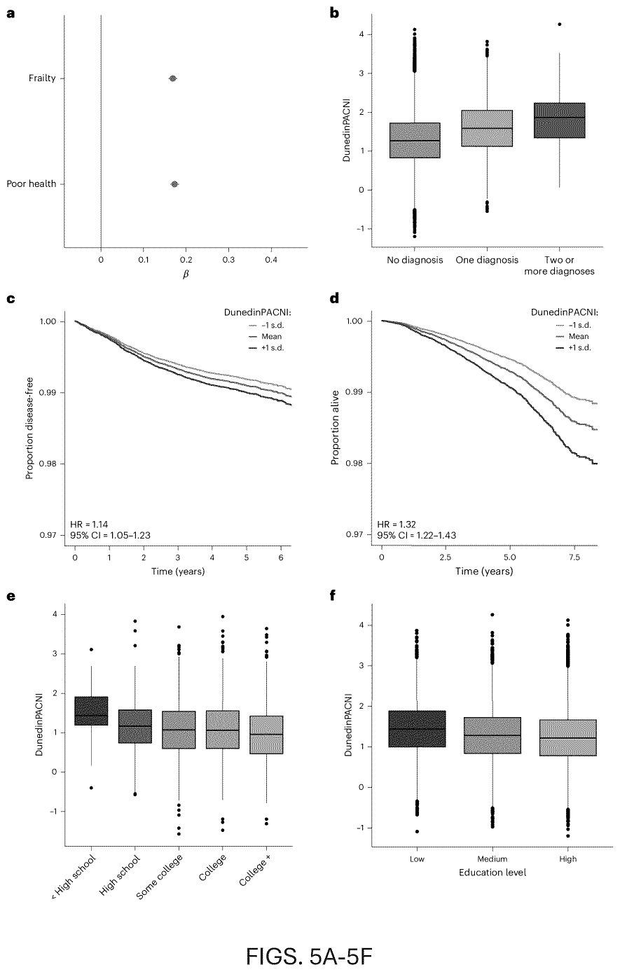

As a measure of aging derived from longitudinal assessments of multiple biomarkers, DunedinPACNI should capture instances of declining health across all organ systems, not just the brain. To test this hypothesis, we used UKB to map DunedinPACNI scores onto measures of frailty, subjective overall health, incident aging-related chronic diseases and all-cause mortality. We used the Fried Frailty Index to quantify the degree of vulnerability to common stressors associated with aging-related decline in energy reserves and functioning. When treating index scores as a continuous measure ranging from 0 to 5, with higher scores indicating greater frailty52,53, we found that participants with faster DunedinPACNI were frailer (n=42,583; β=0.17, P<0.001, 95% CI=0.16 to 0.18). Participants with faster DunedinPACNI also self-reported poorer overall health (n=42,235; β=−0.17, P<0.001, 95% CI=−0.18 to −0.16; FIG. 5A), which predicts mortality even independently of objective health measures54. These associations were not driven by individuals with early cognitive decline or high genetic risk for AD (FIG. 10 and Table 2).

Similar patterns emerged when considering clinical diagnoses of chronic aging-related diseases, including myocardial infarction, chronic obstructive pulmonary disease, dementia and stroke. Participants with a lifetime prevalence of one of these chronic diseases had faster DunedinPACNI compared to those with no diagnoses (β=0.19, P<0.001, 95% CI=0.16 to 0.23). Participants with a lifetime prevalence of two or more chronic diseases had faster DunedinPACNI than those with a single chronic disease (β=0.25, P<0.001, 95% CI=0.12 to 0.38) and those with no chronic disease (β=0.44, P<0.001, 95% CI=0.31 to 0.57; FIG. 5B). Extending beyond contemporaneous associations, we assessed whether faster DunedinPACNI at baseline predicted future myocardial infarction, chronic obstructive pulmonary disease, dementia or stroke in UKB participants who were diagnosis-free at the time of scanning (n=40,753). A total of 827 participants reported a new diagnosis of at least one of these aging-related chronic diseases over a maximum follow-up period of 9.7 years after scanning (that is, baseline). Consistent with the contemporaneous associations, healthy participants with faster DunedinPACNI at baseline were more likely to be later diagnosed with chronic aging-related diseases (HR=1.14, P<0.001, 95% CI=1.05 to 1.23; FIG. 5C), that is, those in the top 10% had an 18% or greater increased risk of developing a chronic disease compared to participants with average DunedinPACNI. These associations were not driven by individuals with early cognitive decline or high genetic risk for AD (FIG. 10 and Table 2).

Given the increased mortality rates among people with chronic aging-related diseases, we asked if baseline DunedinPACNI scores predicted all-cause mortality. Of the 42,583 UKB participants included in our dataset, 757 died over the follow-up period after their baseline MRI scan. Participants with faster baseline DunedinPACNI scores died earlier (HR=1.32, P<0.001, 95% CI=1.22 to 1.43; FIG. 5D), that is, those in the top 10% were at least 41% more likely to die compared to participants with average DunedinPACNI. These associations were not driven by individuals with early cognitive decline or high genetic risk for AD (FIG. 10 and Table 2). Taken together, these findings suggest that DunedinPACNI is useful for gauging general physical health and assessing the risk for future chronic disease and death.

### DunedinPACNI Reflects Social Gradients of Health Inequities

People who are less advantaged in their socioeconomic position experience a wide range of chronic diseases and earlier mortality55,56,57; DunedinPACNI should reflect such gradients of health inequities. We used information about educational attainment and income to test this prediction. Faster DunedinPACNI was observed for participants who either had fewer years of formal education (ADNI: β=−0.10, P<0.001, 95% CI=−0.15 to −0.05]; UK Biobank: β=−0.09, P<0.001, 95% CI=−0.10 to −0.08) or lower income (UKB: β=−0.06, P<0.001, 95% CI=−0.07 to −0.05), reflecting the expected socioeconomic health gradient (FIG. 5E, 5F). These associations were not driven by individuals with early cognitive decline or high genetic risk for AD (FIG. 10).

### DunedinPACNI Generalizes to a Latin American Sample

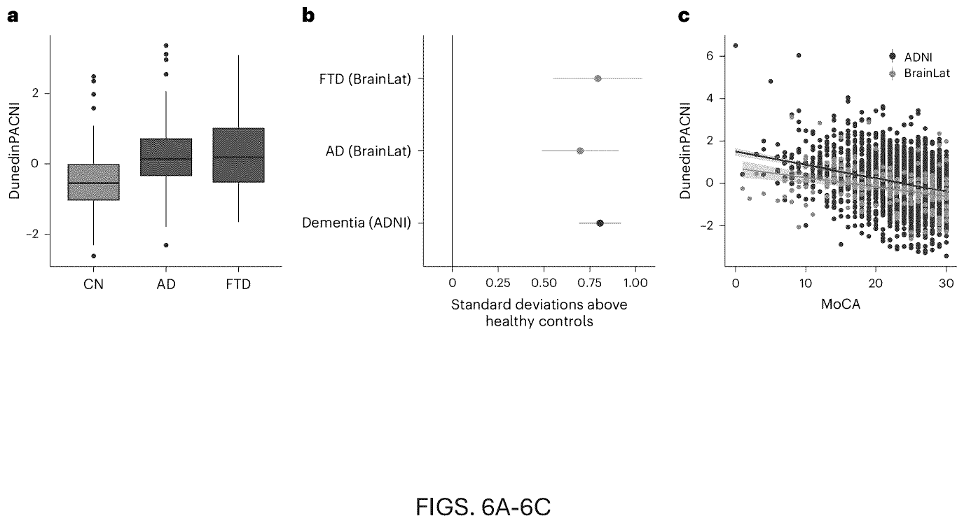

Brain-based predictive algorithms often fail when applied to groups that demographically differ from the training sample58. Most cognitive and brain aging studies are collected in high-income countries in North America and Europe; this may limit the generalizability of brain-based models to people in low-income and middle-income countries59,60,61. Latin Americans are underrepresented in biomedical research60,62 and may experience distinct social and environmental influences on brain aging compared to people in high-income countries in North America and Europe60. To test the generalizability of DunedinPACNI in Latin Americans, we calculated DunedinPACNI scores in a sample of 369 adults from Argentina, Chile, Colombia, Mexico and Peru from the BrainLat dataset63. A total of 162 BrainLat participants were diagnosed with AD, 84 were diagnosed with behavioral variant frontotemporal dementia (FTD) and 123 were healthy controls. When controlling for age and sex, BrainLat participants with AD and FTD had faster DunedinPACNI scores compared to healthy controls (AD: β=0.70, P<0.001, 95% CI=0.48 to 0.91; FTD: β=0.79, P<0.001, 95% CI=0.55 to 1.04; FIG. 6A). Notably, these effect sizes are comparable to the difference between participants with dementia and CN participants in ADNI (FIG. 6B). In addition, 191 BrainLat participants also completed the Montreal Cognitive Assessment (MoCA). In this subset, BrainLat participants with faster DunedinPACNI scores had poorer scores on the MoCA (β=−0.35, P<0.001, 95% CI=−0.49 to −0.20). Notably, this effect was similar to the association between DunedinPACNI and MoCA scores in ADNI participants (β=−0.32, P<0.001, 95% CI=−0.38 to −0.25; FIG. 6C).

DunedinPACNI is Distinct from Common Measures of Brain Aging

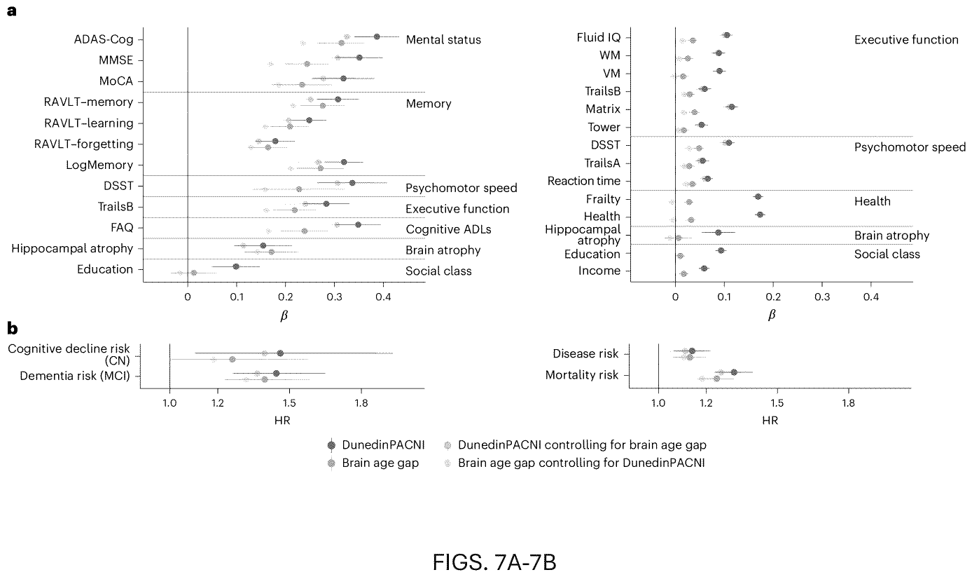

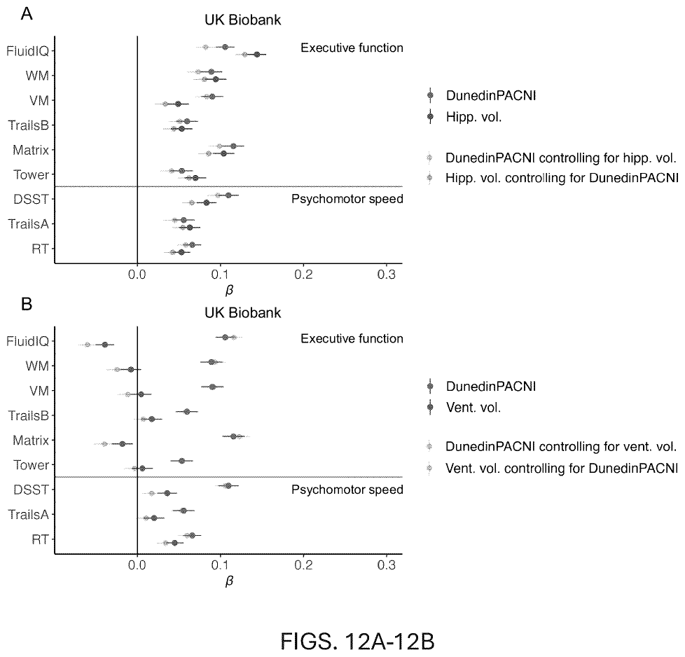

Lastly, we compared DunedinPACNI with existing approaches for measuring aging using brain MRI data. Specifically, we compared the effect sizes for DunedinPACNI from all of the aforementioned analyses in ADNI, UKB and BrainLat with brain age gap generated using brainageR34. We selected this algorithm because of its high accuracy and test-retest reliability compared to other brain age gap algorithms64. DunedinPACNI and brain age gap were only modestly correlated (ADNI: r=0.17, P<0.001; UKB: r=0.31, P<0.001; BrainLat: r=0.32, P<0.001). Compared to brain age gap, the effect sizes for DunedinPACNI were similar or larger across measures of cognitive function, cognitive decline, brain atrophy, frailty, disease risk, mortality and socioeconomic health gradients (FIGS. 7A-7B and FIGS. 12,13; full results are shown in FIGS. 24-25, and Tables 4-9).

Independent models

| | | | | Independent models | | | | Combined models | | | |
|---|---|---|---|---|---|---|---|---|---|---|---|
| | | | | | | 95% | 95% | | | 95% | 95% |
| | | N | | | | CI | CI | | | CI | CI |
| Diagnostic | Brain | In- | Observ- | | | lower | upper | | | lower | upper |
| group | measure | dividuals | ations | beta | p-value | bound | bound | beta | p-value | bound | bound |
| MCI vs. CN | DonedinPACNI | 1466 | 5064 | 0.27 | <0.001 | 0.18 | 0.35 | 0.23 | <0.001 | 0.14 | 0.31 |
| MCI vs. CN | Brain age gap | 1466 | 5064 | 0.24 | <0.001 | 0.14 | 0.34 | 0.19 | <0.001 | 0.09 | 0.29 |
| Dementia | DunedinPACNI | 1201 | 3333 | 0.81 | <0.001 | 0.66 | 0.92 | 0.68 | <0.001 | 0.57 | 0.80 |
| vs.CN | | | | | | | | | | | |
| Dementia | Brain age gap | 120 | 3333 | 0.68 | <0.001 | 0.57 | 0.80 | 0.52 | <0.001 | 0.40 | 0.64 |
| vs.CN | | | | | | | | | | | |

Table 4: Associations between DunedinPACNI, brain age gap, and cognitive gap in ADNI. Note: Combined models included both DunedinPACNI and brain age gap in the same model. Therefore results from the combined models show the effect for DunedinPACNI while controlling for brain age gap, and vice versa. Abbreviations: ADNI=Alzheimer's Disease Neuroimaging Initiative, CI=confidence interval, CN=cognitively normal, MCI=mild cognitive impairment.

| | | | Independent models | | | | Combined models | | | |
|---|---|---|---|---|---|---|---|---|---|---|
| | | | | | 95% Cl | 95% CI | | | 95% CI | 95% CI |
| | | | | | lower | upper | | | lower | upper |
| Dataset | Brain model | N | beta | p-value | bound | bound | beta | p-value | bound | bound |
| UK Biobank | DunedinPACNI | 4601 | −0.09 | <0.001 | −0.12 | −0.05 | −0.09 | <0.001 | −0.12 | −0.06 |
| UK Biobank | Brain age gap | 4601 | −0.01 | 0.646 | −0.04 | 0.02 | 0.01 | 0.491 | −0.02 | 0.04 |
| ADNI | DunedinPACNI | 1300 | −0.15 | <0.001 | −0.21 | −0.10 | −0.11 | <0.001 | −0.17 | −0.05 |
| ADNI | Brain age gap | 130 | −0.17 | <0.001 | −0.23 | −0.12 | −0.14 | <0.001 | −0.20 | −0.09 |

Table 5: Associations between DunedinPACNI, brain age gap and hippocampal atrophy. Note: Combined models included both DunedinPACNI and brain age gap in the same model. Therefor, results from the combined models show the effect for DunedinPACNI while controlling for brain age gap, and vice versa. Abbreviations: ADNI=Alzheimer's Disease Neuroimaging Initiative, CI=confidence interval.

| | | | | Independent models | | | | Combined models | | | |
|---|---|---|---|---|---|---|---|---|---|---|---|
| | | | | | | 95% CI | 95% CI | | | 95% CI | 95% CI |
| | | | | | | lower | upper | | | lower | upper |
| Outcome | Dataset | Brain variable | N | HR | p-value | bound | bound | HR | p-value | bound | bound |
| Cog. Decline | ADNI | DunedinPACNI | 622 | 1.49 | 0.005 | 1.12 | 1.97 | 1.40 | 0.023 | 1.05 | 1.86 |
| (among CN) | | | | | | | | | | | |
| Cog. Decline | ADNI | Brain age gap | 622 | 1.26 | 0.044 | 1.01 | 1.58 | 1.19 | 0.146 | 0.94 | 1.49 |
| (among CN) | | | | | | | | | | | |
| Dementia | ADNI | DunedinPACNI | 701 | 1.44 | <0.001 | 1.26 | 1.65 | 1.37 | <0.001 | 1.19 | 1.56 |
| (among MCI) | | | | | | | | | | | |
| Dementia | ADNI | Brain age gap | 701 | 1.40 | <0.001 | 1.23 | 1.59 | 1.32 | <0.001 | 1.16 | 1.51 |
| (among MCI) | | | | | | | | | | | |
| Chronic disease | UK Biobank | DunedinPACNI | 40753 | 1.14 | 0.001 | 1.05 | 1.23 | 1.11 | 0.010 | L.03 | 1.20 |
| Chronic disease | UK Biobank | Brain age gap | 40753 | 1.13 | 0.001 | 1.05 | 1.21 | 1.11 | 0.006 | 1.03 | 1.19 |
| Mortality | UK Biobank | DunedinPACNI | 42583 | 1.32 | <0.001 | 1.22 | 1.43 | 1.26 | <0.001 | 1.16 | 1.37 |
| Mortality | UK Biobank | Brain age gap | 42583 | 1.24 | <0.001 | 1.15 | 1.34 | 1.18 | <0.001 | 1.10 | 1.28 |

Table 6: Associations between DunedinPACNI, brain age gap and risk for cognitive decline, chronic disease, and death. Note: Combined models included both DunedinPACNI and brain age gap in the same model. Therefore results from the combined models show the effect for DunedinPACNI while controlling for brain age gap, and vice versa. Abbreviations: ADNI=Alzheimer's Disease Neuroimaging Initiative, CI=confidence interval, CN=cognitively normal, HR=hazard ratio, MCI=mild cognitive impairment.

| | | | | Independent models | | | | Combined models | | | |
|---|---|---|---|---|---|---|---|---|---|---|---|
| | | | | | | 95% CI | 95% CI | | | 95% CI | 95% CI |
| | | | | | | lower | upper | | | lower | upper |
| Outcome | Dataset | Brain model | N | beta | p-value | bound | bound | beta | p-value | bound | bound |
| Years education | UK Biobank | DunedinPACNI | 38297 | −0.09 | <0.001 | −0.10 | −0.08 | −0.10 | <0.001 | −0.11 | −0.08 |
| Years education | UK Biobank | Brain age gap | 38297 | −0.01 | 0.053 | −0.02 | 0.00 | −0.01 | 0.053 | −0.02 | 0.00 |
| Years education | ADNI | DunedinPACNI | 1734 | −0.10 | <0.001 | −0.15 | 0.05 | −0.10 | <0.001 | −0.15 | −0.05 |
| Years education | ADNI | Brain age gap | 1734 | −0.01 | 0.608 | −0.06 | 0.04 | 0.01 | 0.570 | −0.04 | 0.06 |
| Income | UK Biobank | DunedinPACNI | 38297 | −0.06 | <0.001 | −0.07 | −0.05 | −0.06 | <0.001 | −0.07 | −0.05 |
| Income | UK Biobank | Brain age gap | 38297 | −0.02 | 0.001 | −0.03 | 0.01 | −0.02 | 0.001 | −0.03 | −0.01 |

Table 7: Associations between DunedinPACNI, brain age gap and socioeconomic status. Note: Combined models included both DunedinPACNI and brain age gap in the same model. Therefore results from the combined models show the effect for DunedinPACNI while controlling for brain age gap, and vice versa. Abbreviations: ADNI=Alzheimer's Disease Neuroimaging Initiative, CI=confidence interval.

| | | | Independent models | | | | Combined models | | | |
|---|---|---|---|---|---|---|---|---|---|---|
| | | | | | 95% CI | 95% CI | | | 95% CI | 95% CE |
| Diagnostic | | | | | lower | upper | | | lower | upper |
| group | Brain variable | N | beta | p-value | bound | bound | beta | p-value | bound | bound. |
| AD vs. CN | DunedinPACNI | 285 | 0.70 | <0.001 | 0.49 | 0.91 | 0.38 | <0.001 | 0.17 | 0.58 |
| FTD vs. CN | DunedinPACNI | 207 | 0.79 | <0.001 | 0.55 | 1.04 | 0.51 | <0.001 | 0.28 | 0.75 |
| AD vs. CN | Brain age gap | 285 | 0.81 | <0.001 | 0.60 | 1.02 | 0.53 | <0.001 | 0.32 | 0.73 |
| FTD vs. CN | Brain age gap | 207 | 0.71 | <0.001 | 0.46 | 0.96 | 0.39 | 0.002 | 0.15 | 0.63 |

Table 8: Associations between DunedinPACNI, brain age gap and dementia in the BrainLat sample. Note: Combined models included both DunedinPACNI and brain age gap in the same model. Therefore results from the combined models show the effect for DunedinPACNI while controlling for brain age gap, and vice versa. Abbreviations: AD=Alzheimer's dementia, CI=confidence interval, CN=cognitively normal, FTD=frontotemporal dementia.

| | | | Independent models | | | | Combined models | | | |
|---|---|---|---|---|---|---|---|---|---|---|
| | | | | | 95% CL | 95% CI | | | 95% CI | 95% CI |
| | | | | | lower | upper | | | lower | upper |
| Measure | Brain variable | N | beta | p-value | bound | bound | beta | p-value | bound | bound |
| MoCA | DunedinPACNI | 191 | −0.35 | <0.001 | −0.49 | −0.20 | −0.21 | 0.015 | −0.38 | −0.04 |
| MoCA | Brain age gap | 191 | −0.33 | <0.001 | −0.46 | −0.20 | −0.24 | 0.002 | −0.39 | −0.09 |

Table 9: Associations between DunedinPACNI, brain age gap and cognitive status in the BrainLat sample. Note: Combined models included both DunedinPACNI and brain age gap in the same model. Therefore results from the combined models show the effect for DunedinPACNI while controlling for brain age gap, and vice versa. Abbreviations: CI=confidence interval, MoCA=Montreal Cognitive Assessment.

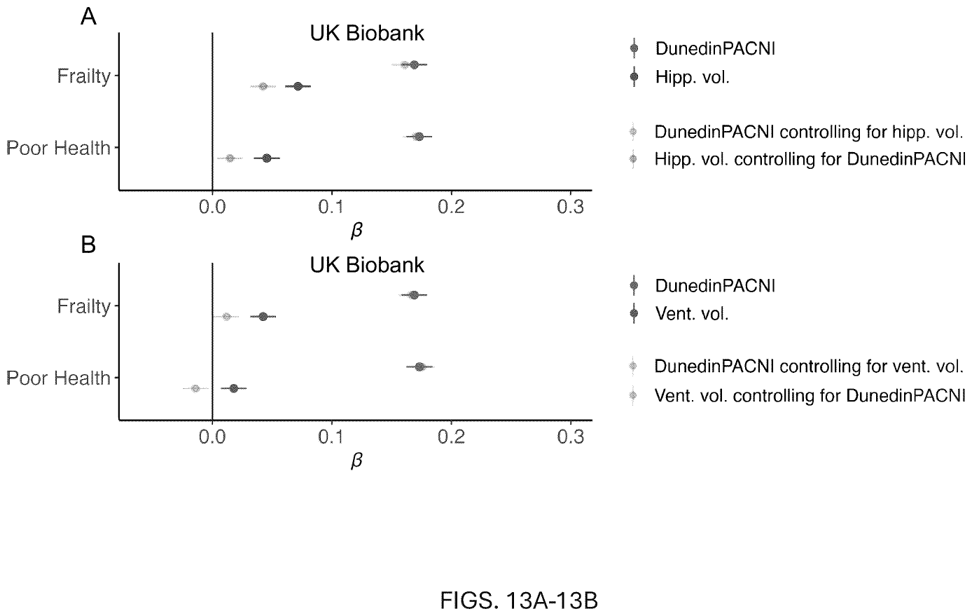

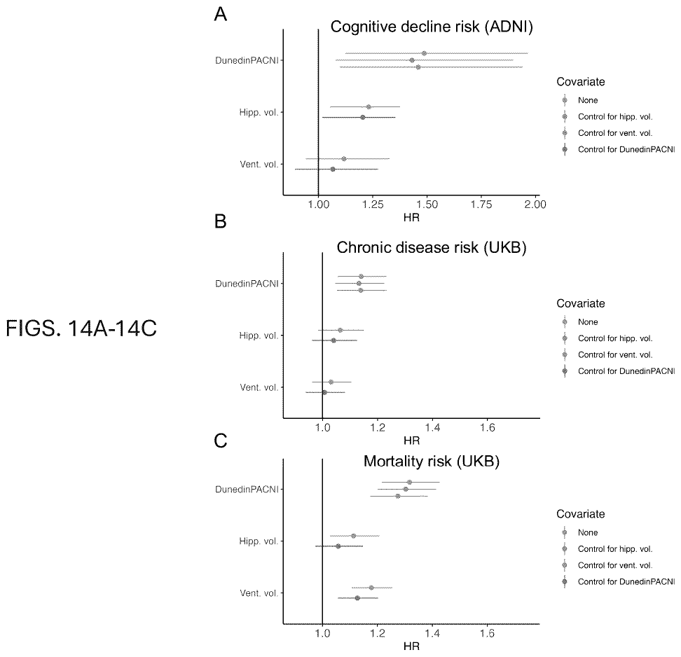

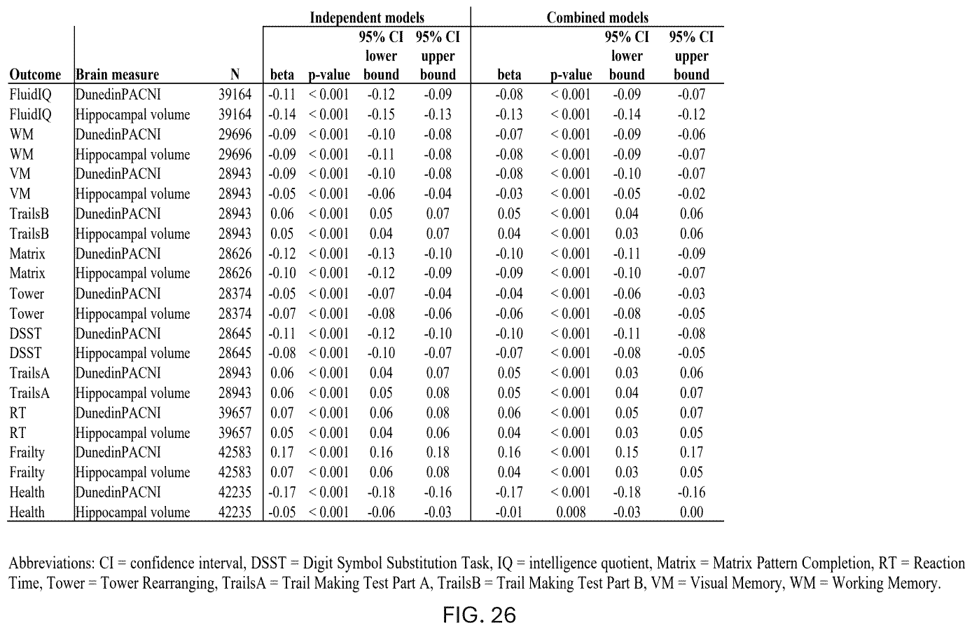

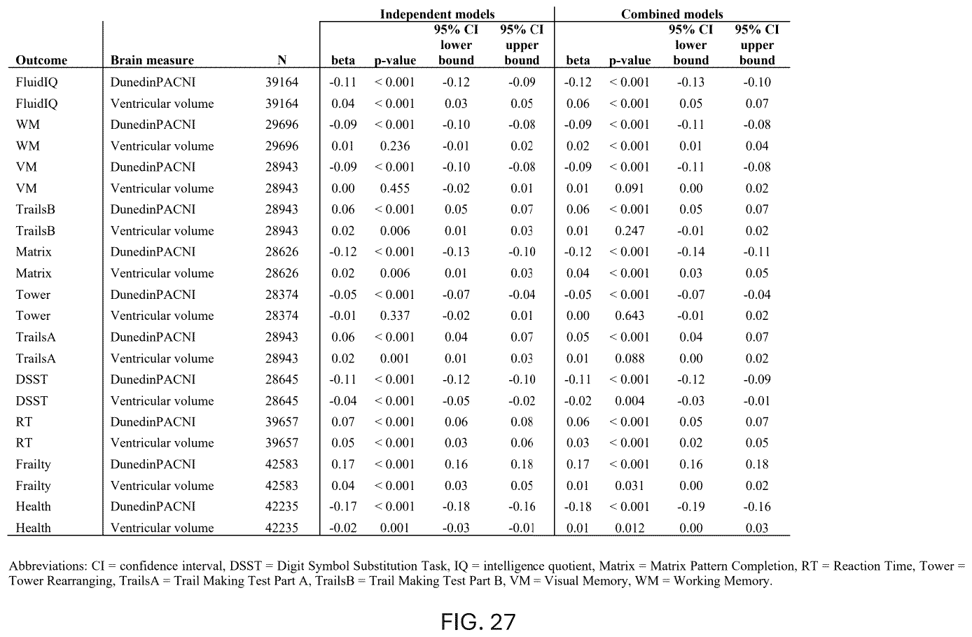

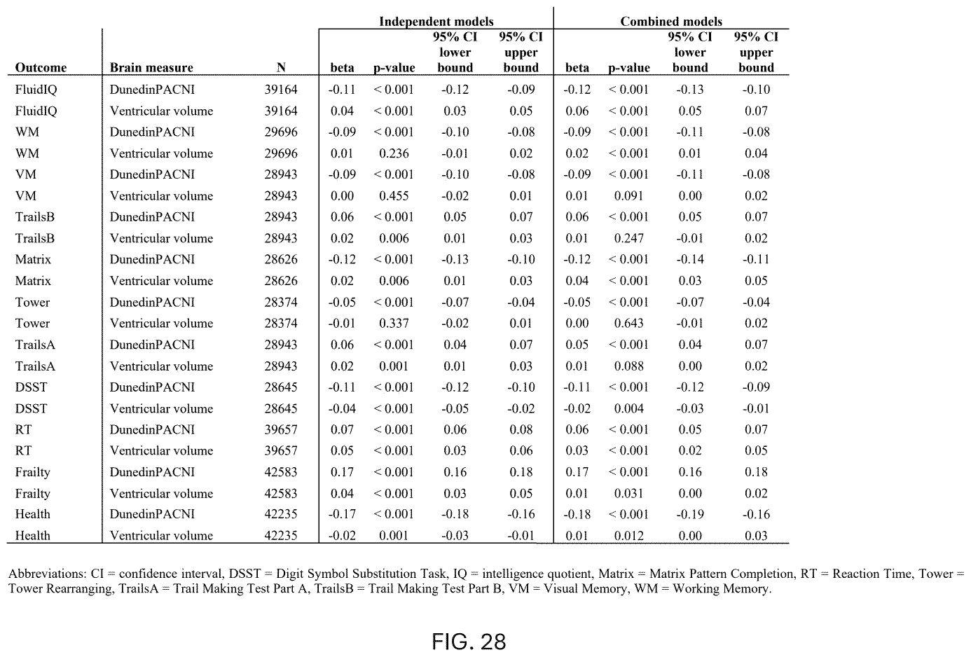

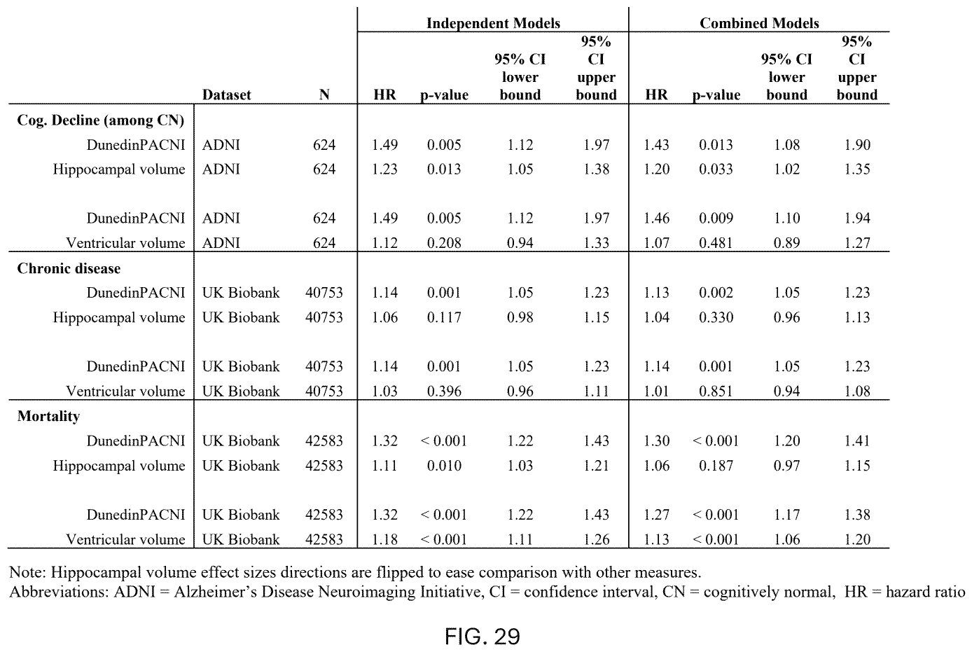

In addition, we investigated how DunedinPACNI differs from commonly reported MRI-based measures of brain aging, namely hippocampal volume and ventricular volume. To test this, we compared the effect sizes for DunedinPACNI with the effect sizes for bilateral hippocampal volume and bilateral ventricular volume in UKB and in CN participants in ADNI. In both datasets, we observed that a faster DunedinPACNI was generally more strongly and more consistently associated with poor cognition, poor health and frailty, as well as risk of dementia, disease and mortality. Furthermore, DunedinPACNI explained incremental variance in these outcomes over and above hippocampal volume and ventricular volume alone (FIGS. 12A-12B, 13A-13B, 14A-14C, 26, 27, and 28).

### Discussion

DunedinPACNI is an accurate and reliable measure of how fast a person is aging derived from a single brain MRI scan. Using 50,106 scans from people ranging from 22 to 98 years old across the HCP, ADNI, UKB and BrainLat datasets, we demonstrate that people with faster DunedinPACNI had not only worse cognitive and brain health (that is, poorer cognition, faster hippocampal atrophy and greater dementia risk) but also worse general health (that is, greater frailty, poorer self-reported health, greater risk of chronic disease and mortality). This indicates that patterns of aging detected during midlife are clinically useful among people in advanced age, including people with neurodegenerative disease. Furthermore, DunedinPACNI showed evidence of generalization to a sample of Latin American adults with and without dementia. Aging remains a construct in search of measurement tools4,5, and DunedinPACNI represents a next-generation measure of aging that is distinct from existing approaches.

Currently, DunedinPACNI and general aging biomarkers are research tools that require further validation before potential translation to the clinic5. Nonetheless, we believe that DunedinPACNI and other biomarkers of general aging have several promising uses that differ from, and are complementary to, biomarkers of specific diseases (for example, hippocampal atrophy and AD72 or high blood pressure and stroke73). For example, biomarkers of general aging could help identify a broad risk for many age-related diseases earlier in the lifespan, when individuals may benefit most from health and antiaging intervention23. Biomarkers of general aging could also help establish a person's prognosis after the onset of a specific disease or establish how interventions or insults may change how fast a person is aging25,74,75. Lastly, biomarkers of general aging are necessary to mechanistically link exposures (for example, low socioeconomic status) to aging or to link the rate of aging to outcomes such as increased disease risk4. While detecting or diagnosing a specific disease requires a proximal, specific biomarker of that disease, biomarkers of general aging could be used to measure the distal risk for a broad array of diseases. This difference allows for clinical applications of biomarkers of aging such as DunedinPACNI alongside biomarkers of specific diseases.

Several unique features of the Dunedin Study contribute advantages to DunedinPACNI compared to other biomarkers of aging. First, DunedinPACNI was developed in a cohort of people all born in the same year and studied at the same ages throughout their lives, thereby avoiding biases that are introduced by differences in historical exposures across generations and across time. Second, DunedinPACNI was trained on 19 biomarkers that were each assessed over nearly two decades and thus are not influenced by short-term illnesses that can cause aberrant biomarker signals at a single assessment. Third, DunedinPACNI was derived from participants followed from birth to age 45 years-before the onset of chronic, aging-related diseases that cause divergence from typical trajectories of aging. Other biomarkers of aging are typically derived from multiage samples in which many of the older members already have chronic diseases that have altered their body and brain, so those measures may inadvertently repackage disease instead of aging. Fourth, because the Dunedin Study cohort is a population representative cohort with very low attrition and mortality rates, DunedinPACNI does not suffer from oversampling of healthy volunteers, attrition bias (that is, people with worse health being more likely to drop out) or survivor bias (that is, people with worse health dying earlier). Indeed, our results align with prior research showing that DunedinPACE, which like DunedinPACNI was trained on the longitudinal Pace of Aging, is associated with dementia, morbidity and mortality20,22,23. Our results, alongside the fast-growing literature on DunedinPACE, suggest that these unique design characteristics of the Dunedin Study make it a powerful training sample for longitudinal biomarkers of aging.

The scope of geroscience has rapidly expanded with the proliferation of omic clocks that can measure how fast people age10. DunedinPACNI is poised to further this growth by allowing individual differences in the rate of longitudinal aging to be estimated from a single noninvasive MRI scan that can be collected in just a few minutes. Indeed, the requisite MRI data to estimate DunedinPACNI have already been collected in many psychiatric, neurological and brain health cohorts, from tens of thousands of research participants across the lifespan and around the world. DunedinPACNI offers an opportunity to enrich such studies and deepen our understanding of the causes of individual differences in the rate of longitudinal aging, including genetics76, childhood adversities77, environmental exposures (for example, lead78,79) and lifestyle factors (for example, physical inactivity, social isolation80). DunedinPACNI may also be adopted as a surrogate endpoint to accelerate our ability to develop, prioritize and evaluate potential antiaging interventions that slow aging and prevent disease81,82.

### Methods

This research complies with all relevant ethical regulations; all study protocols were approved by the relevant ethical review boards. The specific ethical review boards are detailed in the description of each dataset. The premise and analysis plan for this study were preregistered. All analyses and code were checked for accuracy by an independent analyst. Analyses were conducted on data collected through the Dunedin Study, HCP, ADNI, UKB and BrainLat. The details of each study and dataset are described below.

### The DunedinPACNI Algorithm is Publicly Available at GitHub

(//github.com/etw11/DunedinPACNI). All scripts used in the analyses presented in this article are publicly available at GitHub (//github.com/etw11/WhitmanElliott_2024). See also Nature Aging 2025 5:1619-1636 and Whitman et al., bioRxiv Apr. 23, 2025 available at //doi.org/10.1101/2024.08.19.608305 both of which are incorporated herein in their entirety.

### Data Sources

### Dunedin Study

Participants are members of the Dunedin Study, a longitudinal investigation of health and behavior in a population-representative birth cohort. The 1,037 participants (91% of eligible births, 48% female) were all people born between April 1972 and March 1973 in Dunedin, New Zealand, who were residents in the province and who participated in the first assessment at age 3 years19. The cohort represented the full range of socioeconomic status in the general population of New Zealand's South Island and, as adults, matched the New Zealand National Health and Nutrition Survey on key adult health indicators (for example, BMI, smoking and general practitioner visits) and the New Zealand Census of citizens of the same age on educational attainment19,83. Study members are primarily of New Zealand European ethnicity; 8.6% reported Māori ethnicity at age 45 years.

General assessments were performed at birth as well as ages 3, 5, 7, 9, 11, 13, 15, 18, 21, 26, 32 and 38 years, and most recently (completed April 2019) at age 45 years, when 938 of the 997 living study members (94.1%) participated. At each assessment, study members were brought to the Dunedin Study Research Unit at the University of Otago for interviews and examinations. In addition, staff provided standardized ratings; informant questionnaires were sent to people who the study members nominated as people who knew them well, and administrative records were searched. The Dunedin Study was approved by the University of Otago Ethics Committee and study members gave written informed consent before participating.

### MRI

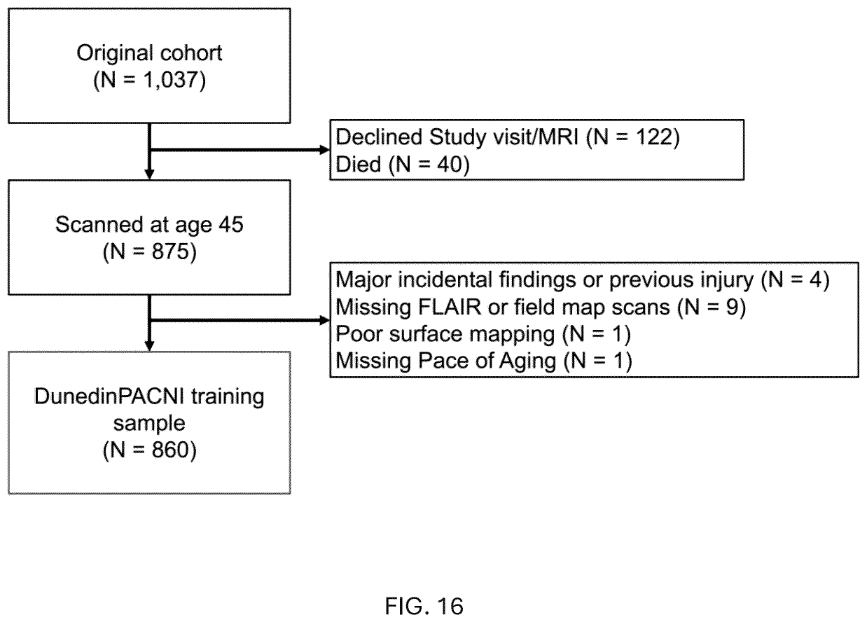

As a component of the assessments at age 45 years, study members were scanned using a Siemens MAGNETOM Skyra (Siemens Healthineers) 3T scanner equipped with a 64-channel head/neck coil at the Pacific Radiology Group imaging center in Dunedin, New Zealand. High-resolution T1-weighted images were obtained using an MP-RAGE sequence with the following parameters: repetition time (TR)=2,400 ms; echo time (TE)=1.98 ms; 208 sagittal slices; flip angle, 9°; field of view (FOV), 224 mm; matrix=256×256 px; slice thickness=0.9 mm with no gap (voxel size 0.9×0.875×0.875 mm); and total scan time=6 min and 52 s. Three-dimensional (3D) fluid-attenuated inversion recovery (FLAIR) images were obtained with the following parameters: TR=8,000 ms; TE=399 ms; 160 sagittal slices; FOV=240 mm; matrix=232× 256 px; slice thickness=1.2 mm (voxel size 0.9×0.9×1.2 mm); and total scan time=5 min and 38 s. Additionally, a gradient echo field map was acquired with the following parameters: TR=712 ms; TE=4.92 and 7.38 ms; 72 axial slices; FOV=200 mm; matrix=100×100 px; slice thickness=2.0 mm (voxel size 2 mm isotropic); and total scan time=2 min and 25 s. Of the 938 study members seen at phase 45, 63 declined to participate in MRI scanning, that is, 875 study members completed the MRI scanning protocol. Scanned study members did not differ from other living participants in terms of childhood neurocognitive functioning or childhood socioeconomic status (see the attrition analysis in FIGS. 8-9). Of these 875 study members for whom data was available, four were excluded because of major incidental findings or previous injuries (for example, large tumors or extensive damage to the brain or skull), nine because of missing FLAIR or field map scans, one because of poor surface mapping yielding and one because of missing the Pace of Aging variable. This yielded a final training sample of 860 study members (see FIG. 16 for the inclusion details).

Structural MRI data were processed using FreeSurfer v.6.0 (ref. 36). Specifically, T1-weighted images were processed and refined with 3D FLAIR images using the recon-all pipeline.

### Pace of Aging

Participants' pace of biological aging was measured as changes in 19 biomarkers of study members' cardiovascular, metabolic, pulmonary, kidney, immune and dental systems across ages 26, 32, 38 and 45 years. This measure quantifies participants' rate of aging in year-equivalent units of physiological decline per chronological year. The average participant experienced 1 year of physiological decline per year, that is, a mean (s.d.) Pace of Aging of 1 (0.3) 2. See the ‘Statistical analysis’ section for more details.

### Physical Functioning

One-legged balance was measured using the unipedal stance test as the maximum time achieved across three trials of the test with eyes closed84,85,86. Gait speed (meters per second) was assessed with the 6-m-long GAITRite Electronic Walkway (CIR Systems) with 2-m acceleration and 2-m deceleration before and after the walkway, respectively. Gait speed was assessed under three walking conditions: usual gait speed (walk at a normal pace from a standing start, measured as a mean of two walks) and two challenge paradigms, dual-task gait speed (walk at a normal pace while reciting alternate letters of the alphabet out loud, starting with the letter ‘A’, measured as a mean of two walks) and maximum gait speed (walk as fast as safely possible, measured as a mean of three walks). Gait speed was correlated across the three walk conditions87. To increase reliability and take advantage of the variation in all three walk conditions (usual gait and the two challenge paradigms), we calculated the mean of the three highly correlated individual walk conditions to generate our primary measure of composite gait speed. The step in place test was measured as the number of times the right knee was lifted to mid-thigh height (measured as the height half-way between the knee cap and the iliac crest) in 2 min at a self-directed pace88,89. Chair rises were measured as the number of stands with no hands completed in 30 s from a seated position88,90. Handgrip strength was measured for each hand (elbow held at 90°, upper arm held tight against the trunk) as the maximum value achieved across three trials using a Jamar digital dynamometer91,92. Analyses using handgrip strength controlled for BMI. Visuomotor coordination was measured as the time to completion of the grooved pegboard test. Scores were reversed so that higher values corresponded to better performance. Physical limitations were measured with the RAND 36-Item Health Survey 1.0 physical functioning scale. Responses (′limited a lot′, ‘limited a little’, ‘not limited at all’) assessed difficulty with completing several activities (for example, climbing several flights of stairs, walking more than 1 km, participating in strenuous sports). Scores were reversed to reflect physical limitations so that a high score indicates more limitations.

### Subjective Health and Age Appearance

We obtained reports about study members' health and age appearance from three sources: self-reports; informant impressions; and staff impressions. For self-reports, we asked study members about their own impressions of how old they looked: ‘Do you think you look older, younger, or about your actual age?’ Response options were younger than their age, about their actual age, or older than their age. We also asked study members to rate their age perceptions in years: ‘How old do you feel?’ For informant impressions, informants who knew a study member well (94% response rate) were asked: ‘Compared to others their age, do you think they (the study member) look younger or older than others their age? Response options were: ‘much younger’, ‘a bit younger’, ‘about the same’, ‘a bit older’ or ‘much older’. For staff impressions, four members of the Dunedin Study unit staff completed a brief questionnaire describing each study member. To assess age appearance, staff used a seven-item scale to assign a ‘relative age’ to each study member (1=young-looking, 7=old-looking). Correlations between self-ratings, informant ratings and staff ratings ranged from 0.34 to 0.52. All reporters rated the study member's general health using the following response options: excellent, very good, good, fair or poor. Correlations between self-ratings, informant ratings and staff ratings ranged from r=0.48 to r=0.55.

### Cognitive Functioning

The Wechsler Adult Intelligence Scale, Fourth Edition was administered at age 45 years, yielding the adult IQ. In addition to full-scale IQ, the Wechsler Adult Intelligence Scale, Fourth Edition yields indexes of four specific cognitive functional domains: processing speed; working memory; perceptual reasoning; and verbal comprehension. The Wechsler Intelligence Scale for Children-Revised was administered at ages 7, 9 and 11 years. To increase the baseline reliability, the three scores were averaged, yielding the childhood IQ. We measured cognitive decline by studying adult IQ scores after controlling for childhood IQ scores. We focused on change in overall IQ given evidence that aging-related slopes are correlated across all cognitive functions, indicating that research on cognitive decline may be best focused on a highly reliable summary index, rather than focused on individual functions93.

### Facial Age

Facial age was based on two measurements of perceived age by an independent panel of eight people. First, age range was assessed by an independent panel of four raters, who were presented with standardized (non-smiling) digital facial photographs of study members when they were 45 years old. Raters, who were kept blind to the actual age of study members, used a Likert scale to categorize each study member into a 5-year age range (that is, from 20 to 24 years old and up to 70+ years old). Interrater reliability was 0.77. The scores for each study member were averaged across all raters. Second, relative age was assessed by a different panel of four raters, who were told that all photos were of people aged 45 years old. These raters then used a 7-item Likert scale to assign a ‘relative age’ to each participant (that is, 1=‘young-looking’ to 7=‘old-looking’). Interrater reliability was 0.79. The measure of perceived age at 45 years (that is, facial age) was derived by standardizing and averaging age range and relative age scores. HCP

The HCP is a publicly available dataset that includes 1,206 participants with extensive MRI data49. HCP data access is managed by the WU-Minn HCP consortium. All participants provided written informed consent. Specifically, we used data from 45 participants who completed the scan protocol a second time (with a mean interval between scans of approximately 140 days) allowing for the calculation of test-retest reliability. All participants were free of current psychiatric or neurological illness and were between 25 and 35 years of age. The mean age of the HCP test-retest sample analyzed was 30.3 years (s.d.=3.3 years, range=22-35 years) at the first time point.

### MRI

Structural MRI data were analyzed using the HCP minimal preprocessing pipeline94. Briefly, T1-weighted images were processed using a custom FreeSurfer recon-all pipeline optimized for structural MRI with a higher resolution than 1 mm isotropic. Details on HCP MRI data acquisition have been described elsewhere94.

### ADNI

The primary goal of ADNI is to test whether serial MRI, positron emission tomography, other biological markers and clinical and neuropsychological assessments can be combined to measure the progression of neurodegeneration in participants with MCI, AD and CN older adults (adni.loni.usc.edu) 95. Cognitive and diagnostic data were downloaded on 12 Jun. 2022. MRI data curated from the Alzheimer's Disease Sequencing Project collection were downloaded on 7 Dec. 2023. ADNI was approved by the institutional review boards of all the participating institutions. All participants provided written informed consent.

### MRI

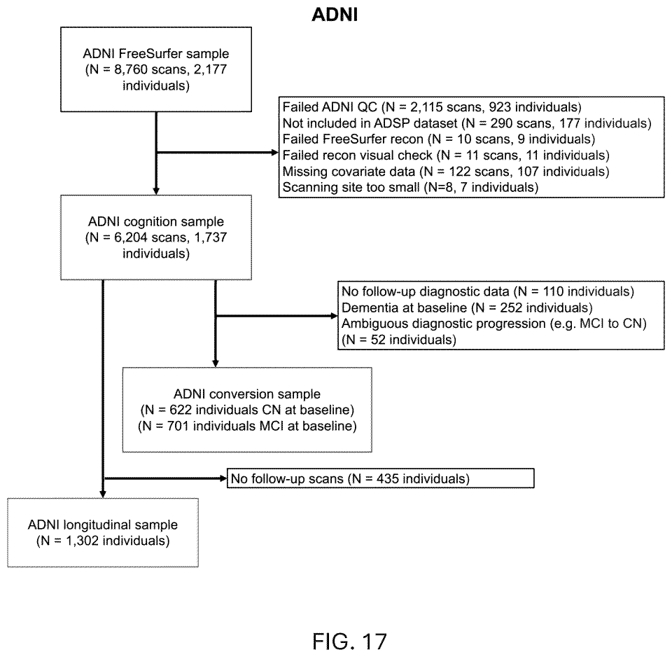

T1-weighted scans were collected using either 1.5T or 3T scanners. MRI acquisition parameters varied across ADNI sites and waves; however, the targets for acquisition were isotropic 1-mm3 voxels96. Raw T1-weighted images were processed using longitudinal FreeSurfer v.6.0. Scans were excluded for low quality if they did not have a quality control rating of ‘pass’ from the ADNI investigators or if segmentation failed visual inspection. Scans were also excluded if participants were missing demographic data, such as age, sex or diagnosis (FIG. 17). Further details on the MRI methods in ADNI can be found at adni.loni.usc.edu.

### Cognitive and Behavioral Functioning

ADNI participants completed several cognitive and behavioral assessments at the time of scanning. The ADAS-Cog is a structured scale that evaluates memory, reasoning, language, orientation, ideational praxis and constructional praxis97. Delayed word recall and number cancellation are included in addition to the 11 standard ADAS items98. The test is scored for errors, ranging from 0 (best performance) to 85 (worst performance). The MMSE is a screening instrument that evaluates orientation, memory, attention, concentration, naming, repetition and comprehension, and ability to create a sentence and to copy two overlapping pentagons99. The MMSE is scored as the number of correctly completed items ranging from 0 (worst performance) to 30 (best performance). The MoCA is designed to detect people at the MCI stage of cognitive dysfunction100. The scale ranges from 0 (worst performance) to 30 (best performance). The RAVLT is a list learning task that assesses learning and memory. On each of five learning trials, 15 unrelated nouns are presented orally at the rate of one word per second and immediate free recall of the words is elicited. After a 30-min delay filled with unrelated testing, free recall of the original 15-word list is elicited. Both immediate recall and percentage forgotten are used. The Log Mem tests I and II (delayed paragraph recall) are from the Wechsler Memory Scale-Revised. Free recall of one short story is elicited immediately after being read aloud to the participant and again after a 30-min delay. The total bits of information recalled after the delay interval (maximum score=25) are analyzed. The Trail Making Test, Part B, consists of 25 circles, either numbered (one through 13) or containing letters (A through L). Participants connect the circles while alternating between numbers and letters (for example, A to 1, 1 to B, B to 2, 2 to C). Time to complete (300 s maximum) is the primary measure of interest. The FAQ is a self-report measure of instrumental ADLs, such as preparing meals, performing chores, keeping a schedule and traveling outside of one's neighborhood101. Each unique cognitive testing measure was paired with the participant's most temporally proximate brain scan within 6 months of cognitive testing.

### Cognitive Status

ADNI participants were classified into CN, MCI or dementia groups by ADNI study physicians based on subjective memory complaints, multiple neurocognitive and behavioral assessment scores, and level of impairment in ADLs. Complete diagnostic criteria can be found at adni.loni.usc.edu. Each individual scan was categorized according to the most temporally proximate cognitive diagnosis received by that participant.

### Education

Education level was measured according to self-reported years of education. For the purposes of visualization in FIG. 5E, participants were grouped according to the following thresholds: less than high school: <12 years; high school: 12 years; some college: 12-15 years; college: 16 years; more than college: >16 years.

### UKB

UKB is a UK population-based prospective study of 502,486 participants between the ages of 40 and 69 at baseline assessment102. We analyzed data from 42,583 participants who underwent brain MRI. The data used in these analyses were downloaded in April 2023. UKB was approved by the North West Multi-centre for Research Ethics Committee. All participants provided written informed consent.

### MRI

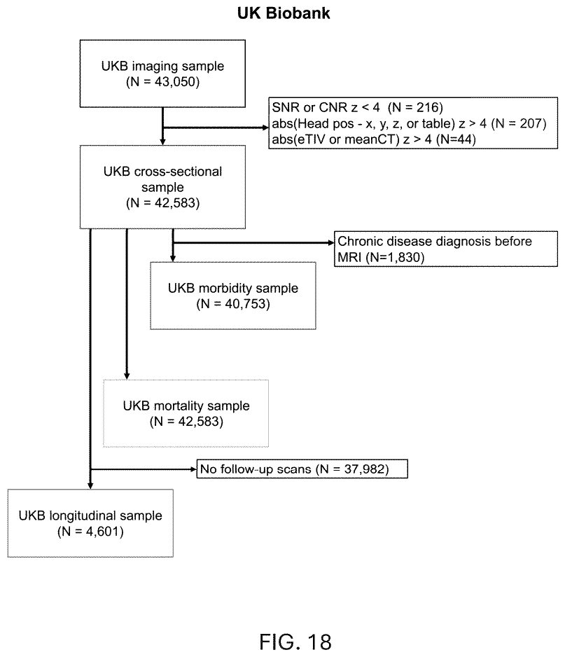

MRI methods for UKB have been described in detail elsewhere103. Briefly, MRI data were collected using three identical 3T Siemens Skyra scanners with a 32-channel Siemens head coil. T1-weighted images were obtained using a 3D MP-RAGE with the following parameters: TR=2,000 ms; inversion time=880 ms; 208 sagittal slices, matrix=256×256 px; slice thickness=1 mm with no gap; and total scan time=4 min and 52 s. Our study made use of imaging-derived phenotypes generated using an image-processing pipeline developed and run on behalf of UKB103. As part of this pipeline, raw T1-weighted images were processed using the cross-sectional FreeSurfer v.6.0. All brain measures used in the cross-sectional analyses presented in this study were derived from the outputs of this FreeSurfer pipeline. We excluded UKB participants with a very low signal-to-noise ratio and highly unusual summary morphometrics indicative of low-quality reconstruction (FIG. 18).

To measure change in hippocampal volume in the subset of UKB participants with longitudinal MRI data, we reprocessed all T1-weighted images for this subset of participants using the longitudinal FreeSurfer v.6.0 pipeline104. This allowed us to avoid the known biases that can be introduced by different processing stages of the longitudinal pipeline in different hardware and software environments. Specifically, we reprocessed both time points of each participant's T1-weighted scans with the cross-sectional recon-all pipeline105. Then, we built an unbiased within-participant template106 using robust, inverse, consistent registration 107 and reprocessed each T1-weighted scan through the automated longitudinal pipeline104.

### Cognitive Functioning

UKB participants completed a battery of cognitive tests at the time of MRI. We investigated cognitive functioning using the following measures: Reaction Time (field ID=20023), Fluid Intelligence (field ID=20016), Numeric Memory (field ID=4282), Trail A (field ID=6348) and Trail B (field ID=6350), symbol digit substitution (field ID=23324), Tower Rearranging (field ID=21004) and Matrix Completion (field ID=6373). The details of these cognitive tests have been described elsewhere108.

### Frailty and Self-Reported Health

To further investigate aging-related health, we used the Fried Frailty Index53. Briefly, the Fried Frailty Index is based on meeting the criteria for declining functioning across five domains: unintentional weight loss; exhaustion; weakness; physical inactivity; and slow walking speed. Index scores range from 0 to 5, with higher scores indicating greater frailty 109. During their imaging visit, UKB participants were also asked to rate their overall health as ‘poor’, ‘fair’, ‘good’ or ‘excellent’. We used these ratings to investigate self-reported overall health (field ID=2178).

### Disease and Mortality Records

To assess the influence of DunedinPACNI on aging-related disease and mortality risk in UKB, we used variables from algorithmically defined health outcomes. Briefly, algorithmically defined outcomes are generated by combining information from baseline assessments (self-reported medical conditions, operations and medications) with linked data from hospital admissions and death registries. Because of the relatively small number of aging-related disease diagnoses at the follow-up, we defined aging-related morbidity as being diagnosed with myocardial infarction (field ID=42000), chronic obstructive pulmonary disease (field ID=42016), dementia (field ID=42018) or stroke (field ID=42006). Furthermore, we defined the risk of chronic disease as the emergence of one or more of these diagnoses among participants who were healthy at the time of scanning (that is, baseline). Mortality was quantified during the follow-up from death records (field ID=40000).

### Education, Income and Ethnicity

To test the association between DunedinPACNI and socioeconomic gradients of health, we tested whether UKB participants differed in DunedinPACNI scores as a function of educational attainment and household income. We grouped participants into three categories according to their self-reported educational qualifications (field ID=6138) following prior work110. Specifically, these groups were: high (college or university degree); medium (A/AS level or equivalent or O level/GCSE or equivalent); and low (none of these). We also tested whether UKB participants differed in DunedinPACNI scores as a function of household income (field ID=738).

We also conducted sensitivity analyses while restricting the UKB sample to either only low-income or only non-White participants. We considered participants as having a low income if they reported making less than £18,000 per year in household income. We considered participants to be non-White if they did not report their ethnic background (field ID=21000) as ‘any other White background’, ‘British’, ‘do not know’, ‘Irish’, ‘prefer not to answer’ or ‘White’.

### BrainLat

BrainLat is a multimodal neuroimaging dataset of patients with neurodegenerative diseases and healthy adult controls collected in Argentina, Chile, Colombia, Mexico and Peru63. We analyzed neuroimaging data from 368 individuals who were either cognitively healthy or diagnosed with AD or behavioral variant FTD. The BrainLat study was approved by the institutional ethical boards of each recruitment site. All participants, or their legal representatives, provided written informed consent.

### MRI

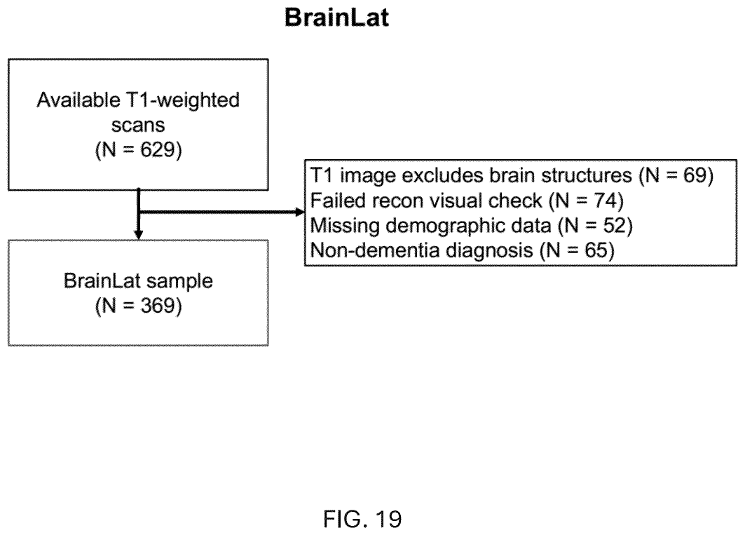

The MRI methods for BrainLat have been described in detail elsewhere63. Briefly, T1-weighted MP-RAGE scans were collected on either 1.5 or 3T scanners. Acquisition parameters varied across sites, but scans most frequently had isometric 1-mm3 voxels. Scans were downloaded and then processed using FreeSurfer v.6.0. Participants were excluded if they failed the automated FreeSurfer quality metrics or a visual quality check of segmentation output (FIG. 19).

### Diagnostic Classification

All participants included could speak fluent Spanish and had adequate visual and auditory capacity for testing. Participants were classified as CN if they had a modified clinical dementia rating of 0, an MMSE score above 25 and lacked a history of substance abuse, and neurological or psychiatric disorders. Patients were classified into the AD or FTD groups according to the National Institute of Neurological Disorders and Stroke-Alzheimer Disease and Related Disorders working group for probable AD or probable behavioral variant FTD. Diagnosis was supported using appropriate MRI or positron emission tomography imaging when needed63.

### Cognitive Status

BrainLat participants were evaluated with the MoCA. The MoCA is designed to detect people at the MCI stage of cognitive dysfunction 100. The scale ranges from 0 (worst performance) to 30 (best performance).

### Statistical Analyses

### Pace of Aging

The derivation of Pace of Aging has been described elsewhere1,2. Briefly, we measured a panel of the following 19 biomarkers (FIG. 1A) at ages 26, 32, 38, and 45 years: BMI, waist/hip ratio, HbAlc, leptin, blood pressure (mean arterial pressure), cardiorespiratory fitness (VO2max), forced vital capacity ratio (FEV1/FVC), FEV1, total cholesterol, triglycerides, HDL, lipoprotein (a), apolipoprotein B100/A1 ratio, eGFR, blood urea nitrogen, hsCRP, white blood cell count, mean periodontal AL and the number of dental-caries-affected tooth surfaces (tooth decay). To calculate each study member's Pace of Aging, we first transformed the biomarker values to a standardized scale. For each biomarker at each wave, we standardized values according to the age 26 distribution. Next, we calculated each study member's slope for each of the 19 biomarkers using a mixed-effects growth model that regressed the biomarker's level on age. Finally, we combined information from the 19 slopes of the biomarkers using a unit-weighting scheme. We calculated each study member's Pace of Aging as the sum of age-dependent annual changes in biomarker z-scores. Biomarker standardization was performed separately for men and women.

### DunedinPACNI

A schematic of DunedinPACNI model development can be found in FIG. 1. We trained an elastic net regression model to estimate the Pace of Aging from structural neuroimaging phenotypes in 860 Dunedin Study members at age 45 years (for the attrition analysis and inclusion criteria see FIGS. 8, 9, and 16). We selected 315 FreeSurfer measures as predictors from the following categories: regional CT, regional cortical SA, regional cortical GMV, regional cortical GWR and ‘ASEG’ volumes (that is, regional subcortical GMV, ventricular volumes and bilateral volume of white matter hypointensities). All cortical data were parcellated according to the Desikan-Killiany Atlas111. Note that although many ADNI scans do not pass quality control (FIG. 17), FreeSurfer is a robust segmentation method, especially in healthy individuals112. Four phenotypes from the ‘ASEG’ volumes were excluded because of insufficient variance in the Dunedin Study (left and right white matter hypointensities, left and right non-white matter hypointensities). Model training was performed using the caret package in R. We conducted a grid search across a range of a and 2 values. We used 100 repetitions of tenfold cross-validation to estimate model performance in held-out participants. The effect of sex was regressed from the Pace of Aging before model training. To prevent information leak during cross-validation, we regressed sex from each training set and applied the resulting β weights to each test set. This approach ensured that our model only used information from the training set, including covariate regression, when calculating predictions in each test set. We selected optimal tuning parameters according to the highest variance explained and lowest mean absolute error. The optimal tuning parameters were α=0.214 and λ=0.100. Using these parameters, we fitted the model to the entire n=860 sample.

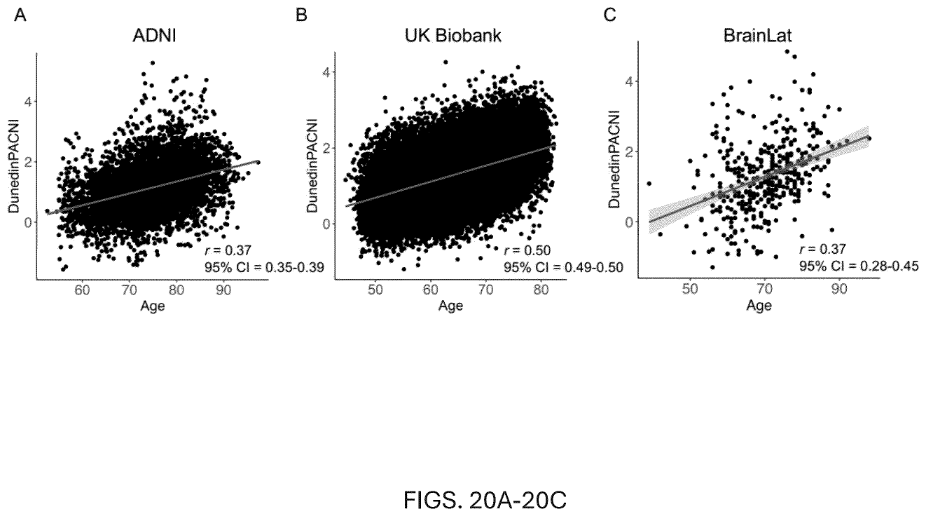

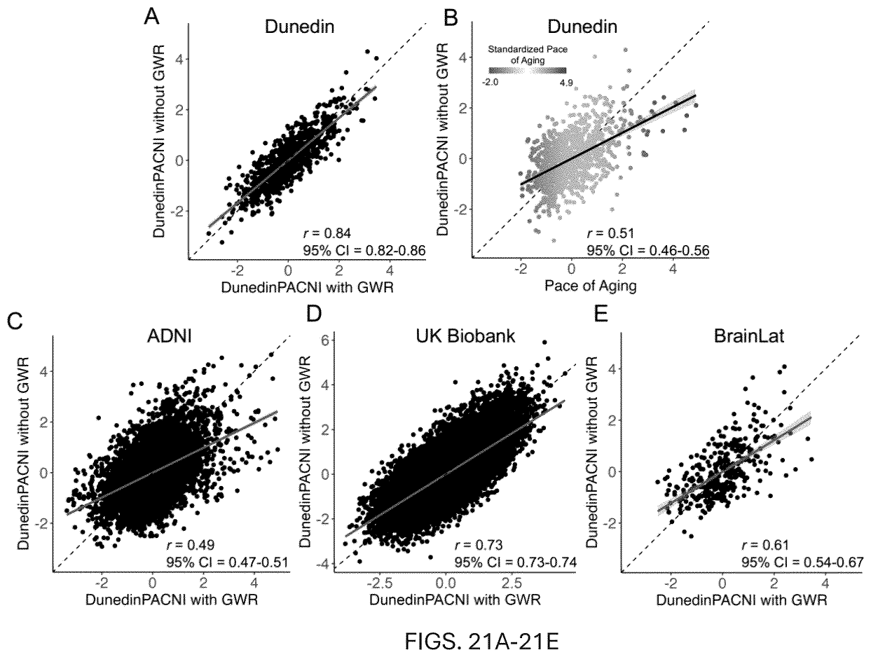

To generate DunedinPACNI scores in HCP, ADNI, UKB and BrainLat participants, we applied the regression weights from the DunedinPACNI model to FreeSurfer-derived phenotypes in each dataset and summed the products and model intercept. In ADNI, UKB and BrainLat, DunedinPACNI scores were correlated with chronological age (ADNI: r=0.37; UKB: r=0.50; BrainLat: r=0.37; FIGS. 20A-20C).

In addition, we conducted the same procedure again without GWR because this measure is not always distributed in public datasets. We observed slightly reduced model accuracy when GWR was not included. DunedinPACNI estimates without GWR phenotypes showed excellent test-retest reliability in HCP. DunedinPACNI estimates were similar with and without GWR phenotypes in ADNI, UKB and BrainLat.

### Brain Age Gap

We submitted raw T1-weighted images from ADNI, UKB and BrainLat to the publicly available brainageR algorithm (v.2.1). This model, which has been described in detail elsewhere113, is trained to predict chronological age in a sample of healthy, cognitively unimpaired individuals aged 18-92 years. This algorithm was selected because it generates highly reliable estimates among published algorithms64. Briefly, brainageR is estimated by first segmenting and normalizing T1-weighted images using SPM12. Next, coefficients derived from a Gaussian process regression model predicting chronological age in a training dataset (n=2,001) are applied to morphometric features from brain segmentations to predict participants' chronological age. Brain age gap was subsequently estimated by subtracting actual chronological age from predicted age113. Notably, 15 ADNI scans failed the brain age gap pipeline (14 failed visual inspection of segmentation, one error when computing the predicted age). These scans were excluded from all brain age gap analyses, including comparative analyses with DunedinPACNI.

### Dunedin Study Validation Analyses

To first test the validity of DunedinPACNI in the Dunedin Study training sample, we tested for linear associations between DunedinPACNI scores and one-legged balance, gait speed, step in place, chair stands, grip strength, visuomotor coordination, subjective physical limitations, subjective health, cognitive function, child-to-adult cognitive decline and facial aging while controlling for sex. We compared these effect sizes to associations between each of these measures and the original, longitudinal Pace of Aging.

### Test-Retest Reliability

We used the HCP dataset to assess the test-retest reliability of DunedinPACNI. Reliability was quantified using a two-way mixed-effects intraclass correlation coefficient (3,1) with session modeled as a fixed effect, participant as a random effect and the test-retest interval as an effect of no interest114.

### Cognitive and Physical Functioning

We first used linear regression models to test for associations between DunedinPACNI and scores on tests of cognition, physical function and health in ADNI and UKB. All analyses controlled for age and sex. In ADNI, we calculated robust standard errors to account for nonindependence from repeated observations. We also tested the standardized differences in DunedinPACNI scores between three groups based on cognitive status: CN, MCI and dementia. All group difference comparisons controlled for age and sex. We again calculated robust standard errors to account for nonindependence and conduced a sensitivity analysis while controlling for APOE ε4 carriership. We repeated these analyses with brain age gap. Notably, when conducting analyses on the combined effects of DunedinPACNI and brain age gap on cognitive outcomes in ADNI, we restricted the sample to the first time point of each measure. These analyses included only one observation per participant, allowing us to more easily combine effect sizes and CIs.

### Dementia Survival Analysis

We conducted a Cox proportional hazards regression using the baseline DunedinPACNI scores of ADNI participants to predict their probability of cognitive decline or clinical conversion to dementia during the follow-up window. Conversion in CN participants was defined as having a diagnosis of CN at baseline but a diagnosis of MCI or dementia at the end of the follow-up. Conversion in participants with MCI was defined as having a diagnosis of MCI at baseline and a diagnosis of dementia by the end of the follow-up. Participants who had a baseline diagnosis of dementia or transitioned from MCI to CN were not included in this analysis. The analysis controlled for sex, age at baseline and length of the observation window. We investigated the influence of AD genetic risk on these results by conducting all analyses while additionally controlling for APOE &4 carriership. We repeated these analyses for brain age gap.

### Prediction of Hippocampal Atrophy Rates

We used repeated MRI measurements from ADNI (n=1,302) and UKB (n=4,601) to generate estimates of change in hippocampal GMV. We used longitudinal ComBat on ADNI MRI data to remove differential scanner effects115. Next, using all available time points for each participant, we generated multilevel linear models for bilateral hippocampal volume with random effects for both participant and age. Using these models, we derived trajectories to track change in hippocampal GMV for each participant. We then tested whether each participant's baseline DunedinPACNI scores could predict their subsequent rate of hippocampal atrophy. These analyses controlled for age, sex and length of the observation period. We investigated the influence of AD genetic risk on these results by conducting these analyses while additionally controlling for APOE &4 carriership. We repeated these analyses for brain age gap.

### Morbidity and Mortality Survival Analyses

To investigate the association between DunedinPACNI and morbidity, we used UKB data to calculate the standardized differences in DunedinPACNI scores between three groups based on the number of lifetime chronic disease diagnosis (0, 1, 2+). Next, we conducted a Cox proportional hazards regression using UKB participants' baseline DunedinPACNI scores to predict the onset of a chronic aging-related disease (n=827 emergent diagnoses: myocardial infarction, chronic obstructive pulmonary disease, dementia or stroke) in participants who had never previously received any of these diagnoses at the time of scanning (n=40,753). Similarly, to investigate the association between DunedinPACNI and mortality, we conducted a Cox proportional hazards regression using UKB participants' baseline DunedinPACNI scores to predict death (n=757 deaths). Both models controlled for baseline age, time to onset and sex. We repeated these analyses for brain age gap.

### Socioeconomic Inequality Analyses

To investigate whether DunedinPACNI reflected gradients of socioeconomic inequality57, we first tested for linear relationships between DunedinPACNI and years of education in ADNI and UKB. We also tested for a linear relationship between DunedinPACNI and household income in UKB. These analyses controlled for sex and age. In ADNI, we included only the first MRI observation per participant.

### Replication in a Latin American Sample

To investigate whether DunedinPACNI generalizes to samples of individuals who are underrepresented in neuroimaging research59, we tested whether the degree of acceleration in DunedinPACNI among ADNI participants with dementia was similar in BrainLat participants with dementia. We tested for standardized differences by comparing the AD and FTD groups to the CN group, respectively. We also tested for linear associations between DunedinPACNI and MoCA scores. All analyses in this sample controlled for age and sex. We then compared the magnitude of acceleration among BrainLat participants with dementia to the previously identified acceleration among ADNI participants with dementia. Lastly, we compared the strength of the linear association between DunedinPACNI and MoCA scores in BrainLat participants to the previously identified association in ADNI participants.

Comparison with Hippocampal and Ventricular Volume

We investigated how DunedinPACNI differs from two commonly used MRI-based measures of brain aging: hippocampal volume and ventricular volume. We calculated hippocampal volume as the sum of left and right hippocampal GMV measures derived from FreeSurfer. Likewise, we calculated ventricular volume as the sum of the left and right lateral ventricular volume measures. We first repeated cross-sectional associations with cognition, frailty and poor health in UKB, substituting DunedinPACNI with hippocampal volume or ventricular volume. Next, we conducted this same procedure with our Cox proportional hazards regression models of chronic disease and mortality risk in UKB, and cognitive decline risk among CN ADNI participants. Lastly, we compared coefficients for all analyses while including DunedinPACNI and either hippocampal volume or ventricular volume in the respective models. All analyses controlled for age and sex.

### Example 2—Illustrative Embodiments of the Methods and Systems Disclosed Herein

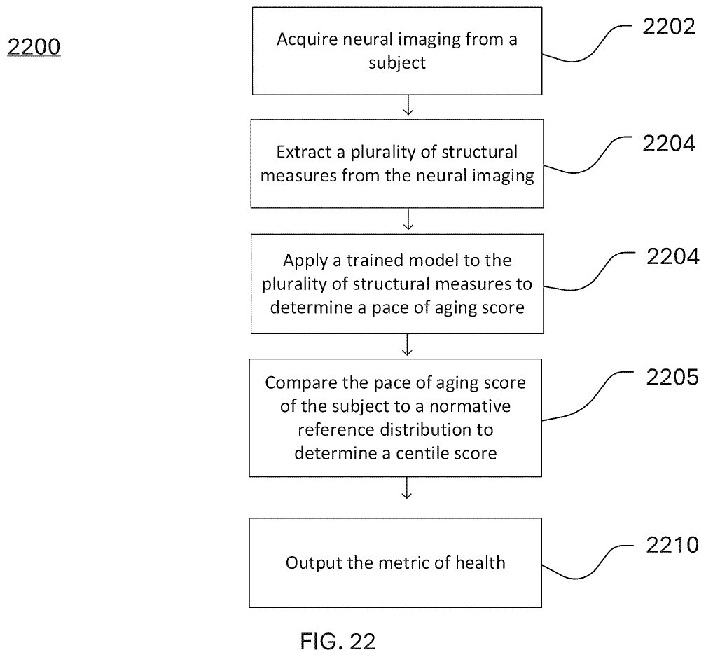

FIG. 22 shows an example process 2200 to provide a metric of aging for a subject is shown. At 2202, brain imaging of the subject may be acquired. At 2204, a plurality of structural features may be extracted. At 2206, a trained model may be applied to the plurality of structural measures from the brain imaging data to determine a pace of aging score. At 2208, the pace of aging score may be compared to a normative reference distribution to determine a centile score, in which the normative reference comprises a plurality of pace of aging scores corresponding to a plurality of control subjects. At 2210, a metric of health may be output. The metric of health may include the pace of aging score and the centile score of the subject.

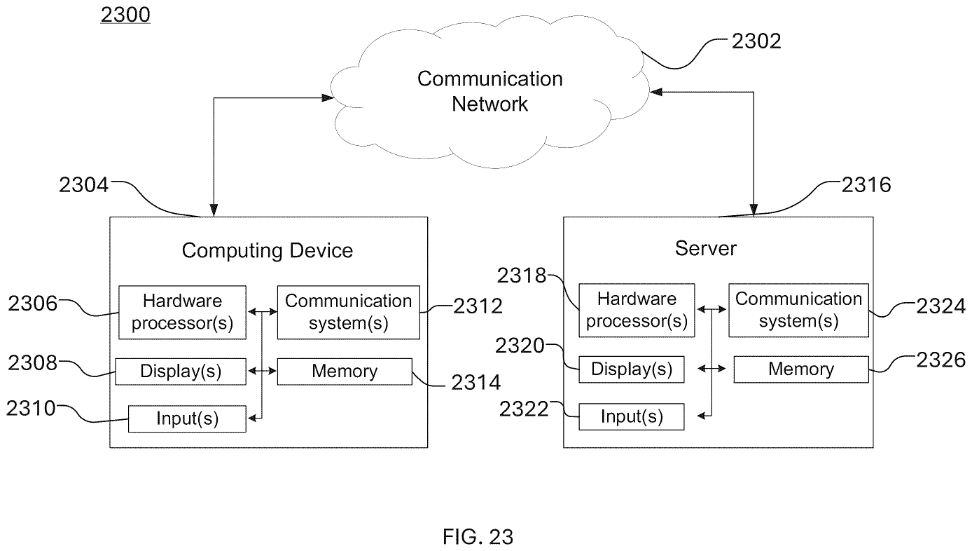

In FIG. 23 an example 2300 of a system (e.g., a data processing system) for estimating a metric of health of a subject is provided. The system 2300 may also be used to predict the likelihood a subject will develop a chronic disorder.

In some embodiments, computing device 2304 and/or server 2316 can be any suitable computing device or combination of devices, such as a desktop computer, a laptop computer, a smartphone, a tablet computer, a wearable computer, a server computer, a virtual machine being executed by a physical computing device, etc. As described herein, system 2300 can present information about the metric of health/pace of aging score/centile score, etc. to a user (e.g., a researcher and/or a physician).

In some embodiments, communication network 2302 can be any suitable communication network or combination of communication networks. In some embodiments, communication network 2302 can be any suitable communication network or combination of communication networks. For example, communication network 2302 can include a Wi-Fi network (which can include one or more wireless routers, one or more switches, etc.), a peer-to-peer network (e.g., a Bluetooth network), a cellular network (e.g., a 4G network, a 5G network, etc., complying with any suitable standard, such as CDMA, GSM, LTE, LTE Advanced, WiMAX, etc.), a wired network, etc. In some embodiments, communication network 2302 can be a local area network, a wide area network, a public network (e.g., the Internet), a private or semi-private network (e.g., a corporate or university intranet), any other suitable type of network, or any suitable combination of networks. Communications links shown in FIG. 23 can each be any suitable communications link or combination of communications links, such as wired links, fiber optic links, Wi-Fi links, Bluetooth links, cellular links, etc.

FIG. 23 additionally shows an example of hardware that can be used to implement computing device 2304 and server 2316 in accordance with some embodiments of the disclosed subject matter. In some embodiments, computing device 2304 can be used to execute one or more sets of instructions to estimate a metric of health of a subject. In other embodiments, computing device 2304 can be used to estimate a likelihood that a subject will develop a chronic disorder.

As shown in FIG. 23 computing device 2304 can include one or more hardware processor 2306, one or more displays 2308, one or more inputs 2310, one or more communications 2312, and/or memory 2314. In some embodiments, processor 2306 can be any suitable hardware processor or combination of processors, such as central processing unit, a graphics processing unit, etc. In some embodiments, display 2308 can include any suitable display devices, such as a computer monitor, a touchscreen, a television, etc. In some embodiments, inputs 2310 can include any suitable input device and/or sensors that can be used to receive user input, such as a keyboard, a mouse, a touchscreen, a microphone, etc.

In some embodiments, communication systems 2312 can include any suitable hardware, firmware, and/or software for communicating information over communication network 2302 and/or any other suitable communication networks. For example, communications systems 2312 can include one or more transceivers, one or more communication chips and/or chip sets, etc. In a more particular example, communications systems 2312 can include hardware, firmware and/or software that can be used to establish a Wi-Fi connection, a Bluetooth connection, a cellular connection, an Ethernet connection, etc.

In some embodiments, memory 2314 can include any suitable storage device or devices that can be used to store instructions, values, etc., that can be used, for example, by processor 2306 to present content using display 2308, to communicate with server 2316 via communications system(s) 2312, etc.

Memory 2314 can include any suitable volatile memory, non-volatile memory, storage, or any suitable combination thereof. For example, memory 2314 can include RAM, ROM, EEPROM, one or more flash drives, one or more hard disks, one or more solid state drives, one or more optical drives, etc. In some embodiments, memory 2314 can have encoded thereon a computer program for controlling operation of computing device 2304. In such embodiments, processor 2306 can execute at least a portion of the computer program to present content (e.g., images, user interfaces, graphics, tables, etc.), receive content from server 2316, transmit information to server 2316, etc.

In some embodiments, server 2316 can include a processor 2318, a display 2320, one or more inputs 2322, one or more communications systems 2324, and/or memory 2326. In some embodiments, processor 2318 can be any suitable hardware processor or combination of processors, such as a central processing unit, a graphics processing unit, etc. In some embodiments, display 2320 can include any suitable display devices, such as a computer monitor, a touchscreen, a television, etc. In some embodiments, inputs 2322 can include any suitable input devices and/or sensors that can be used to receive user input, such as a keyboard, a mouse, a touchscreen, a microphone, etc.

In some embodiments, communications systems 2324 can include any suitable hardware, firmware, and/or software for communicating information over communication network 2302 and/or any other suitable communication networks. For example, communications systems 2324 can include one or more transceivers, one or more communication chips and/or chip sets, etc. In a more particular example, communications systems 2324 can include hardware, firmware and/or software that can be used to establish a Wi-Fi connection, a Bluetooth connection, a cellular connection, an Ethernet connection, etc.

In some embodiments, memory 2326 can include any suitable storage device or devices that can be used to store instructions, values, etc., that can be used, for example, by processor 2318 to present content using display 2320, to communicate with one or more computing devices 2304, etc. Memory 2326 can include any suitable volatile memory, non-volatile memory, storage, or any suitable combination thereof. For example, memory 2326 can include RAM, ROM, EEPROM, one or more flash drives, one or more hard disks, one or more solid state drives, one or more optical drives, etc. In some embodiments, memory 2326 can have encoded thereon a server program for controlling operation of server 2316. In such embodiments, processor 2318 can execute at least a portion of the server program to transmit information and/or content (e.g., results of a trained algorithm, a user interface, etc.) to one or more computing devices 2304, receive information and/or content from one or more computing devices 2304, receive instructions from one or more devices (e.g., a personal computer, a laptop computer, a tablet computer, a smartphone, etc.), etc.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

While the invention has been described above in connection with particular embodiments and examples, the invention is not necessarily so limited, and that numerous other embodiments, examples, uses, modifications and departures from the embodiments, examples and uses are intended to be encompassed by the claims attached hereto.

1. A method for estimating a metric of aging of a subject comprising:
acquiring brain imaging of the subject;
extracting a plurality of structural measures from the brain imaging data;
applying a trained model to the plurality of structural measures to determine a pace of aging score;
comparing the pace of aging score of the subject to a normative reference distribution to determine a centile score, wherein the normative reference comprises a plurality of pace of aging scores corresponding to a plurality of control subjects; and
outputting the metric of aging, wherein the metric of aging comprises the pace of aging score and the centile score of the subject.

2. The method of claim 1, wherein the brain imaging of the subject comprises magnetic resonance imaging (MRI) data and wherein the plurality of structural measures comprises over 300 structural measures.

3. (canceled)

4. The method of claim 1, wherein the plurality of structural measures comprises at least one of regional cortical thickness, surface area, gray matter volume, gray-white matter signal intensity ratio, subcortical thickness, ventricular volumes, or bilateral volume of white matter hypointensities.

5. The method of claim 1, wherein the trained model was trained using longitudinal data from a plurality of subjects, wherein the longitudinal data comprises brain imaging data and information on at least one organ system collected at a plurality of time points and wherein the trained model comprises a regression model comprising at least one of a linear regression, logistic regression, polynomial regression, ridge regression, lasso regression, quantile regression, or elastic net regression.

6. (canceled)

7. The method of claim 5, wherein the trained model comprises an elastic net regression.

8. The method of claim 1, wherein the model is selected based on the sex of the subject.

9. The method of claim 1, wherein the control subjects comprise subjects aged 18-100 years old and who have not been diagnosed with a psychiatric disorder or a neurological disorder.

10. The method of claim 1, wherein the centile score represents the percentile of a subset of the plurality of control subjects with corresponding pace of aging scores below the subject's pace of aging score,
wherein the subset of the plurality of control subjects comprises subjects in a similar age group as the subject and wherein the similar age group comprises ages within 12 months of an age of the subject.

11. (canceled)

12. The method of claim 1, further comprising longitudinal monitoring of the subject, wherein the longitudinal monitoring comprises:
obtaining a second set of brain imaging of the subject at a later point in time;
determining a corresponding updated centile score of the subject by applying the trained model to structural measures extracted from the second set of brain imaging data and comparing a resulting pace of aging score to the normative reference distribution;
quantifying a change in the centile score; and
outputting a second metric of aging and the change in centile score.

13. A method of predicting a subject's likelihood of developing a disorder based on the subject's centile score and pace of aging comprising:
acquiring brain imaging of the subject;
extracting a plurality of structural measures from the brain imaging data;
applying a trained model to the plurality of structural measures to determine the pace of aging score;
comparing the pace of aging score of the subject to a normative reference distribution to determine a centile score, wherein the normative reference distribution comprises a plurality of pace of aging scores corresponding to a plurality of controlled subjects; and
outputting a prediction of the subject's likelihood of developing the disorder, wherein if the centile score is over 50 or the pace of aging is ahead of age and sex matched controls the subject is at increased likelihood of developing the disorder, and;
administering a recommended intervention to mitigate risk of developing the chronic disorder or delay onset of the chronic disorder.

14. (canceled)

15. The method of claim 13, further comprising longitudinal monitoring of the subject, wherein the longitudinal monitoring comprises:
obtaining a second set of brain imaging of the subject at a later point in time;
determining a corresponding updated centile score of the subject by applying the trained model to structural measures extracted from the second set of brain imaging and comparing a resulting pace of aging score to the normative reference distribution;
quantifying a change in the centile score; and
providing an updated prediction of the subject's likelihood of developing the disorder, wherein if the centile score has increased, the centile score is over 50, or the pace of aging is ahead of age and sex matched controls, the subject is at increased likelihood of developing the disorder, wherein if the subject is at increased likelihood of developing the disorder, an intervention is recommended.

16. The method of claim 13, wherein the disorder comprises a chronic disorder and the chronic disorder is selected from the group consisting of stroke, chronic obstructive pulmonary disease, myocardial infarction, or dementia.

17. (canceled)

18. The method of claim 13, wherein the recommended intervention comprises at least one of a change in lifestyle, behavioral therapy, administration of a supplement, administration of a pharmaceutical agent, referral of the subject to a physician for medical intervention, or some combination thereof.

19.-29. (canceled)

30. A system for estimating a metric of aging of a subject, the system comprising:
a processor in communication with a memory, the memory having stored thereon a set of instructions which, when executed by the processor, cause the processor to:
acquire brain imaging data of the subject;
extract a plurality of structural measures from the brain imaging data;
apply a trained model stored in the memory to the plurality of structural measures to determine a pace of aging score;
compare the pace of aging score of the subject to a normative reference distribution to determine a centile score, wherein the normative reference distribution comprises a plurality of pace of aging scores corresponding to a plurality of control subjects, wherein the control subjects comprise subjects aged 18-100 years old who have not been diagnosed with a psychiatric disorder or a neurological disorder; and
output the metric of aging, wherein the metric of aging comprises the pace of aging score and the centile score of the subject.

31. The system of claim 30, wherein the brain imaging data comprise magnetic resonance imaging (MRI) data and wherein the plurality of structural measures comprises over 300 structural measures.

32. (canceled)

33. (canceled)

34. The system of claim 30, wherein the trained model was trained using longitudinal data from a plurality of subjects, the longitudinal data comprising brain imaging data and information on at least one organ system collected at a plurality of time points, wherein the trained model comprises a regression model including at least one of a linear regression, logistic regression, polynomial regression, ridge regression, lasso regression, quantile regression, or elastic net regression and wherein the trained model is selected based on a sex of the subject.

35.-40. (canceled)

41. The system of claim 30, wherein the instructions further cause the processor to perform longitudinal monitoring of the subject by:
obtaining a second brain imaging data set of the subject at a later time;
determining an updated centile score relative to the centile score previously determined for the subject using the trained model and the normative reference distribution;
quantifying a change in the centile score; and
outputting a second metric of aging and the quantified change in the centile score.

42. The system of claim 30 wherein the instructions further cause the processor to output a prediction of the subject's likelihood of developing a disorder,
wherein the subject is determined to have an increased likelihood of developing the disorder if the centile score exceeds 50 or the pace of aging score indicates aging ahead of age and sex matched controls and wherein the disorder comprises a chronic disorder selected from the group consisting of stroke, chronic obstructive pulmonary disease, myocardial infarction, or dementia.

43. The system of claim 42, wherein the instructions further cause the processor to determine a recommended intervention to mitigate a risk of developing the disorder or delay onset of the disorder when the centile score exceeds 50, and output the recommended intervention.

44.-46. (canceled)

47. The system of claim 43, wherein the recommended intervention output by the processor comprises at least one of a lifestyle change, behavioral therapy, administration of a supplement, administration of a pharmaceutical agent, referring the subject to a physician for medical intervention, or a combination thereof.
